(19) United States
(12) Patent Application Publication
(10) Pub. No.: US 2024/0101616 A1
(43) Pub. Date: Mar. 28, 2024

(54) DAP10/12 BASED CARS ADAPTED FOR RUSH

(71) Applicants: INSTITUT CURIE, Paris (FR); INSERM (INSTITUT NATIONAL DE LA SANTE ET DE LA RECHERCHE MEDICALE), Paris (FR); CENTRE NATIONAL DE LA RECHERCHE SCIENTIFIQUE (CNRS), Paris (FR)

(72) Inventors: Franck Perez, Paris (FR); Zelia Gouveia, L'hay les Roses (FR); Sebastian Amigorena, Paris (FR); Gaelle Boncompain, Chatillon (FR)

(73) Assignees: INSTITUT CURIE, Paris (FR); INSERM (INSTITUT NATIONAL DE LA SANTE ET DE LA RECHERCHE MEDICALE), Paris (FR); CENTRE NATIONAL DE LA RECHERCHE SCIENTIFIQUE (CNRS), Paris (FR)

(21) Appl. No.: 18/515,805

(22) Filed: Nov. 21, 2023

Related U.S. Application Data
(63) Continuation of application No. 16/757,409, filed on Apr. 19, 2020.

(30) Foreign Application Priority Data
Oct. 20, 2017 (EP) 17306452.8

(51) Int. Cl. C07K 14/435; C07K 14/705; C07K 16/12; C07K 16/28; C12N 7/00; C12N 15/86

(52) U.S. Cl. CPC C07K 14/435; C07K 14/70517; C07K 14/70575; C07K 16/1292; C07K 16/2896; C12N 7/00; C12N 15/86; C07K 2317/622; C07K 2319/03; C07K 2319/40; C12N 2740/15042

(57) ABSTRACT

A chimeric antigen receptor including: a binding domain, the full DAP 10 protein, the full DAP 12 protein, or a functional variant thereof, and a hook binding domain. Also, a vector system comprising one or more vector including: a nucleic acid comprising a nucleic acid sequence encoding a chimeric antigen receptor and optionally a nucleic acid encoding a hook fusion protein, preferably having a streptavidin core; wherein the nucleic acids are located on the same or on different vectors. Further, a lentiviral vector particles system, host cell and kit including the nucleic acids or vector system, and their use as a medicament, notably for immunotherapy.

### CROSS-REFERENCE TO RELATED APPLICATIONS

This application is continuation application of U.S. application Ser. No. 16/757,409, filed Apr. 19, 2020, which is a national stage entry under 35 U.S.C. § 371 of PCT/EP2018/078931, filed Oct. 22, 2018, which itself claims priority to EP Patent Application Number 17306452.8, filed Oct. 20, 2017; the contents of each of which is incorporated by reference herein in its entirety.

### REFERENCE TO SEQUENCE LISTING

The Sequence Listing submitted herewith via EFS-Web is hereby incorporated by reference in its entirety. The name of the file is IBIO-1611-PCT_ST26.xml, the size of the file is 26,244 bytes, and the date of creation of the file is Nov. 15, 2023.

### FIELD

CAR-T cells are transduced T cells expressing a chimeric antigen receptor construct composed of a binding moiety, typically an antibody-derived fragment (scFv), fused to co-stimulatory motives required to transmit effector signals for T cell activation or other immune cell activation.

### BACKGROUND

Several scFv-CAR T cells are currently tested in clinical trials for the treatment of hematologic malignancies and solid tumors (Brentjens, Davila et al. 2013, June, Maus et al. 2014, Bonini and Mondino 2015, Gross and Eshhar 2016). Despite the promising results obtained using this therapy, several limitations remain that compromise patient safety such that its therapeutic index is still questionable.

For instance CAR-T therapy can lead to autoimmune reactivity and cytokine-associated toxicity and in extreme situations death.

Auto-immune toxicity, also known as “on target/off-tumor” is associated with cross reactivity with normal cells due to a limited amount of specific tumor antigens, or the presence of common antigens in normal cells. This effect has mainly been observed in patients treated with anti-immune checkpoints antibodies (Abs) or CAR-T bearing T cell receptors (TCR) directed towards tumor immune-checkpoints.

The other syndrome is cytokine-associated toxicity, also named cytokine-release syndrome (CRS) caused by over-activation of the immune system (B cells, T cells, and/or natural killer cells) and/or myeloid cells (macrophages, dendritic cells, and monocytes) towards non-specific antigens. This leads to the release of a high quantity of inflammatory cytokines causing over-activation of several immune mechanisms that can be life-threatening with multiple organ failure. This phenomenon has been observed in patient treated with natural or bispecific antibodies as well as with adoptive T-cell therapies against cancer (Wing, Moreau et al. 1996, Winkler, Jensen et al. 1999, Suntharalingam, Perry et al. 2006, Maude, Barrett et al. 2014, Porter, Hwang et al. 2015).

These adverse effects can compromise the patient's life as well as their life quality. It is therefore crucial to improve the safety of those therapies while keeping their efficiency, as well as to develop new therapies with higher efficiency and low or minimized risks for to patient's live and life-quality.

Several strategies are currently being explored in order to overcome such limitations to CAR-cell therapy, however safety concerns remain.

CAR-T cells activated only upon the formation of a complex composed by anti-FITC Abs conjugated with CAR and a “switch” molecule (FITC, fluorescein isothiocyanate) that is linked to an antibody against tumor antigens have been developed (Ma, Kim et al. 2016). The advantage of such system is that the activation of CAR-T cells is achieved only upon recognition of the switch molecule in a dose-dependent manner. They have also shown that this technology can be used for simultaneous targeting of two distinct antigens (e.g. CD19 and CD22). The major limitation is the association between anti-FITC Ab-CAR and FITC-cancer antibodies that was shown to strongly affect the formation of an effective immunological synapse, thus preventing efficient cell activation. In addition, further studies are required to evaluate the immunogenicity of the FITC conjugate and its toxicity.

CAR-T cells have also been optimized (see Rodgers, Mazagova et al. 2016), using a small peptide fused to an anti-tumor antibody as a switch molecule. The activation of CAR-T cells expressing an anti-peptide-directed-CAR occurs only upon recognition of that switch molecule required for formation of the complex between the anti-tumor antibody labelled with the small peptide and the anti-peptide-directed-CAR.

It has also been proposed that activation of CAR-T cells can be achieved by a combinatorial antigen-sensing circuit. Briefly, primarily a synthetic Notch receptor is activated by the recognition of first tumor antigen, leading to the induction of CAR expression that, upon recognition of the second tumor-antigen, activates effector T cells (Roybal, Rupp et al. 2016). In this system, the activation of T cells is restrained to two tumor antigens, presumably increasing the specificity and safety of the T cells. Nevertheless, it is not clear whether this system prevents autoimmune related toxicities. Therefore, the major limitations of this system concern 1) auto-immunogenicity caused by using bacterial and yeast derived transcriptional activators, 2) the two tumor-antigens used in this study, i.e. CD19 and mesothelin, are only co-expressed in certain tumors but not others 3) the use of two antigens, one specific for tumor and another for healthy cells (as they also propose) might lead to toxic effect, when the two types of cells are nearby, leading to their elimination.

A switchable CAR has also been developed using a peptide motif derived from the human nuclear auto-antigen La/SS-B of 10 amino acids (5B9 tag), which is apparently non-immunogenic (Cartellieri, Feldmann et al. 2016). Again, the effectiveness of this therapy needs to be confirmed.

Similarly, a system to control the surface expression of the CAR by small molecules, such as rapamycin and tacrolimus (Juillerat, Marechal et al. 2016) has also been explored. The major drawback of this system is that such molecules are not well tolerated by the human organism, promoting several adverse effects (Juillerat, Marechal et al. 2016). Rapalogs have been developed (in particular by ARIAD pharmaceuticals) but their toxicity in humans and mice remains unknown. Another major drawback of this method is that the rapamycin-binding domains are expressed at the cell surface and may thus create a strong anti-CAR immunity.

In this context it remains highly desirable to provide new CARs whose expression at the cell membrane can be easily controlled, by preventing and/or reactivating the CAR membrane expression.

### SUMMARY

The present invention fulfils this need by providing new CARs comprising:
a binding domain,
the full DAP 10 protein, the full DAP 12 protein, or a functional variant thereof, and
a hook binding domain.

In one embodiment, the hook-binding domain is streptavidin-binding peptide.

Typically, the binding domain comprises a single-chain Fv antibody or a single-domain antibody.

Typically also the CAR can further comprises at least one further activation domain selected from the CD3-ζ chain, the CD28 cytoplasmic domain, the 4-1BB cytoplasmic domain, the OX40 cytoplasmic domain and the ICOS cytoplasmic domain.

The present invention also includes a nucleic acid sequence encoding a chimeric antigen receptor as herein defined.

Said nucleic acid can further comprising a nucleic acid sequence encoding a hook protein; optionally wherein the hook protein comprises a streptavidin domain and an endoplasmic reticulum retention signal.

The present invention also includes a vector system comprising one or more vector comprising:
(a) a nucleic acid comprising a nucleic acid sequence encoding a chimeric antigen receptor as previously defined, and optionally
(b) a nucleic acid encoding a hook fusion protein, preferably comprising a streptavidin, preferably streptavidin core;
wherein the nucleic acids (a) and (b) are located on the same or on different vectors; optionally
wherein the hook-binding domain of chimeric antigen receptor is a streptavidin-binding domain and the hook fusion protein comprises a streptavidin domain; optionally
wherein the hook fusion protein comprises an amino acid sequence corresponding to SEQ ID NO:6.

In one embodiment, the nucleic acids (a) and (b) are located on the same vector; the nucleic acid sequence encoding the hook fusion protein is inserted upstream an IRES sequence or a 2A peptide and the nucleic acid sequence encoding the chimeric antigen receptor is located downstream said IRES sequence or 2A peptide. Preferably also the nucleic acid sequence encoding the hook fusion protein is operably linked to a promoter.

Alternatively, in other embodiment it might be advantageous to use a vector system wherein nucleic acids (a) and (b) are located on separate vectors in order to better control the hook protein expression in the cell and thus the retention/release of the CAR.

The vector system of the invention, can comprises a 02-microglobulin, ubiquitin, MHCI, or MHCII promoter or any viral promoter and notably human viral promoter.

The present invention also includes a viral vector particle system comprising one or more viral vector particle wherein said viral vector particle system comprises a vector system as herein defined.

The present invention also includes an isolated host cell comprising the vector system or the viral particle system as herein defined.

The present invention also includes a kit comprising the chimeric antigen receptor, or the vector system or the viral vector particle system, or the host cell as herein defined.

In the kit of the invention the hook-binding domain of chimeric antigen receptor is typically a streptavidin-binding domain and the hook protein comprises a streptavidin domain, preferably the kit further comprises a streptavidin ligand, which can be selected from biotin or ALis.

The present invention also relates to the chimeric antigen receptor, or the vector system or the viral vector particle system, or the host cell, or the kit as herein defined, for their use as a medicament and in particular in immunotherapy most particularly for inducing an immune response in a human and typically for inducing a controlled immune response in a human.

The surface expression of these new CARs according to the invention can be easily, efficiently and reversibly controlled by a “RUSH” (Retention Using Selective Hook) system. Indeed, the results of the present application show for the first time that the cellular trafficking of DAP10 and DAP12 armed with a binding domain comprising an antibody can be efficiently and reversibly regulated in order to produce a switchable CAR. The present invention therefore provides new “RUSH” CARs, which toxicity can be timely controlled. The regulation of cell the surface expression of the “RUSH” CARs of the invention should notably allow to limit the risk of cytokine storm caused by the intensive immune reaction associated with the massive presence of tumor antigens.

Furthermore, the design of the new CARs of the invention with DAP10 or DAP12 as a scaffold greatly differs from the modular design of the activation domain of conventional CARs. These smaller or “mini” CARs should therefore further improve lentiviral packaging and delivery.

### BRIEF DESCRIPTION OF THE DRAWINGS

FIG. 1: Schematic representation of the bicistronic plasmid encoding the Hook, such as the cytoplasmic hook (Str-Ii, streptavidin (str) fused to the isoform of the human invariant chain of the major histocompatibility complex (Ii; a type II protein) containing an ER retention arginine-based motif at the N-terminal) and the reporter (i.e.: the CAR). The reporter comprises a single variable chain (scFv) fused to a myc tag followed by DAP10 or DAP12 adaptor proteins (type I protein) and short SBP (with 28 aa, instead of the typical 36 aa).

FIGS. 2A-2B: Traffic of RUSH based constructs scFv (CD19)-myc-DAP10-sSBP and scFv(CD19)-myc-DAP12-sSBP using the cytosolic Ii-Str hook. HeLa cells expressing scFv (CD19)-myc-DAP10-sSBP or B) (2A) scFv(CD19)-myc-DAP12-sSBP reporter (anti-myc stained) (2B) non-treated (NT) and treated with biotin and different time point were recorded. Overnight treatment (ON) was performed by adding biotin immediately after adding transduction solution and it is representative of the protein steady state. Nucleus was stained using DAPI.

FIGS. 3A-3B: Percentage of positive transduced HeLa cells with the scFvCD19 CAR juno (scFvCD19-myc-tmCD8-41BB-CD3z-SBP) and scFvCD19-DAP10 (scFvCD19-myc-DAP10-SBP) HeLa cells were transduced with lentiviral vector for scFvCD19 CAR June (scFvCD19-myc-tmCD8-41BB-CD3z-SBP) fused to 2A-Y-FAST: Puromycin (Plamont M-A, Billon-Denis E, Maurin S, et al. Small fluorescence-activating and absorption-shifting tag for tunable protein imaging in vivo. Proceedings of the National Academy of Sciences of the United States of America. PNAS 2016; 113(3):497-502) and scFvCD19-DAP10 CAR (scFvCD19-myc-DAP10-SBP) fused to 2A-Y-FAST:Puromycin. The expression of the constructs was evaluated by flow cytometry using Yellow Fluorescence-Activating and absorption-Shifting Tag (Y-FAST) that is able to react with 4-hydroxy-3-methylbenzylidene-rhodanine (HMBR), activating their green fluorescence (Plamont M-A, Billon-Denis E, Maurin S, et al. Small fluorescence-activating and absorption-shifting tag for tunable protein imaging in vivo. PNAS 2016; 113(3):497-502).

FIG. 4: Control of cytotoxicity using the RUSH-adapted CAR
Cytotoxicity of CAR (anti-CD19-CAR developed by C. June [“June”]) fused to SBP in C-terminal) in the absence or presence of Hook (Str-Ii) at a [2:1] ratio of effector: target, six days post-transduction with lentiviral particles. Cytotoxicity was measured using the xCELLingence system. As positive control, CD8+ T cells expressing the CAR Juno (black solid line) and as negative control CD8+ T cells expressing only the hook (grey dot line) or non-transduced (grey dash line) were used. The presence of SBP will allow the CAR retention by interaction with streptavidin in the Hook. In the absence of biotin CAR transduced with hook is not active. It becomes active upon biotin addition in a similar manner to CAR June alone.

FIGS. 5A-5B: Cell surface expression of new CAR scaffold
A. Several anti-CD19 CAR fused to SBP in C-terminal were designed using different stimulatory domains: the first generation CAR DAP10 with the full stimulatory protein DAP10; the 2 nd generation CAR DAP10-CD3, with an additional CD3 zeta domain fused in the N-terminal to DAP10 followed by SBP and the 1 st generation CAR DAP12 with the full stimulatory protein DAP12 fused to SBP. B. Extracellular expression of the several anti-CD19 CARs designs (as in B) evaluated by FACS. The CAR JUNO and CAR DAP10 are the ones with higher expression for anti-CD19 and the CAR DAP12 the lowest.

FIGS. 6A-6B-6C: Cytotoxicity of anti-CD19 CAR based on conventional 2^{nd} generation and novel scaffolds
Cytotoxicity of the different CAR designs against tumor cell line Raji as measured using the xCELLigence system. Different effectors: target ratio were evaluated; Target 1:1 (FIG. 6A), Target 2:1 (FIG. 6B), Target 5:1 (FIG. 6C). Arrows indicate the time of effector cells addition. CAR DAP10 is the only design with no activity against the tumor cell line Raji at any effector: target ratio. The DAP10-CD3 scaffold shows an efficacy similar to the classical “June” scaffold while the DAP12 scaffold shows a more moderate efficacy at low Effector-target cell ratio but a comparable efficacy at a higher ratio.

### DETAILED DESCRIPTION

### Definitions

Before the present proteins, compositions, methods, and other embodiments are disclosed and described, it is to be understood that the terminology used herein is for the purpose of describing particular embodiments only and is not intended to be exhaustive. It must be noted that, as used in the specification and the appended claims, the singular forms “a,” “an” and “the” include plural referents unless the context clearly dictates otherwise.

The term “comprising” as used herein is synonymous with “including” or “containing”, and is inclusive or open-ended and does not exclude additional, uncited members, elements or method steps.

The full name of individual amino acids are used interchangeably with their standard three letter and one letter abbreviations for each in this disclosure. For the avoidance of doubt, the amino acids are: Alanine (Ala, A), Arginine (Arg, R), Asparagine (Asn, N), Aspartic acid (Asp, D), Cysteine (Cys, C), Glutamic Acid (Glu, E), Glutamine (Gin, Q), Glycine (Gly, G), Histidine (His, H), Isoleucine (lie, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), Valine (Val, V).

As used herein, the term “in vitro” refers to events that occur in an artificial environment, e.g., in a test tube or reaction vessel, in cell culture, in a Petri dish, etc., rather than within an organism (e.g., animal, plant, or microbe). The term “in vivo” refers to events that occur within an organism (e.g., animal, plant, or microbe).

As used herein, the term “isolated” refers to a substance or entity that has been (1) separated from at least some of the components with which it was associated when initially produced (whether in nature or in an experimental setting), and (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The “isolated” products of this invention, including isolated nucleic acids, proteins, polypeptides, and antibodies are not products of nature (i.e., “non-naturally occurring”). Rather, the “isolated” nucleic acids, proteins, polypeptides, and antibodies of this invention are “man-made” products. The “isolated” products of this invention can be “markedly different” or “significantly different” from products of nature. By way of a non-limiting example, the isolated nucleic acids may be purified, recombinant, synthetic, labeled, and/or attached to a solid substrate. Such nucleic acids can be markedly different or significantly different than nucleic acids that occur in nature. By way of further non-limiting example, the “isolated” proteins, polypeptides, and antibodies of this invention may be purified, recombinant, synthetic, labeled, and/or attached to a solid substrate. Such proteins, polypeptides, and antibodies can be markedly different or significantly different from proteins, polypeptides, and antibodies that occur in nature.

The term “peptide” as used herein refers to a short polypeptide, e.g., one that typically contains less than about 50 amino acids and more typically less than about 30 amino acids. The term as used herein encompasses analogs and mimetics that mimic structural and thus biological function.

The term “polypeptide” encompasses both naturally-occurring and non-naturally occurring proteins, and fragments, mutants, derivatives and analogs thereof. A polypeptide may be monomeric or polymeric. Further, a polypeptide may comprise a number of different domains each of which having one or more distinct activities. For the avoidance of doubt, a “polypeptide” may be any length greater two amino acids.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from a cell in which it was synthesized.

The protein or polypeptide can be purified. Preferably, the purified protein or polypeptide is more than 50%, 75%, 85%, 90%, 95%, 97%, 98%, or 99% pure. Within the context of this invention, a purified protein that is more than 50% (etc.) pure means a purified protein sample containing less than 50% (etc.) other proteins. For example, a sample of a protein comprising can be 99% pure if it contains less than 1% contaminating host cell proteins.

The term “polypeptide fragment” as used herein refers to a polypeptide that has a deletion, e.g., an amino-terminal and/or carboxy-terminal deletion compared to a full-length polypeptide, such as a naturally occurring protein. In an embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, or at least 12, 14, 16 or 18 amino acids long, or at least 20 amino acids long, or at least 25, 30, 35, 40 or 45, amino acids, or at least 50 or 60 amino acids long, or at least 70 amino acids long, or at least 100 amino acids long.

The term “fusion protein” refers to a polypeptide comprising a polypeptide or fragment coupled to heterologous amino acid sequences. Fusion proteins are useful because they can be constructed to contain two or more desired functional elements that can be from two or more different proteins. A fusion protein comprises at least 10 contiguous amino acids from a polypeptide of interest, or at least 20 or 30 amino acids, or at least 40, 50 or 60 amino acids, or at least 75, 100 or 125 amino acids. The heterologous polypeptide included within the fusion protein is usually at least 6 amino acids in length, or at least 8 amino acids in length, or at least 15, 20, or 25 amino acids in length. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, “recombinant” may refer to a biomolecule, e.g., a gene or protein, or to a cell or an organism. The term “recombinant” may be used in reference to cloned DNA isolates, chemically synthesized polynucleotides, or polynucleotides that are biologically synthesized by heterologous systems, as well as proteins or polypeptides and/or RNAs encoded by such nucleic acids. A “recombinant” nucleic acid is a nucleic acid linked to a nucleotide or polynucleotide to which it is not linked in nature and/or if it contains any modifications that do not naturally occur to the corresponding nucleic acid in a genome. A “recombinant” protein or polypeptide may be (1) a protein or polypeptide linked to an amino acid or polypeptide to which it is not linked in nature; and/or (2) a protein or polypeptide made by transcription and/or translation of a recombinant nucleic acid. Thus, a protein synthesized by a microorganism is recombinant, for example, if it is synthesized from an mRNA synthesized from a recombinant nucleic acid present in the cell. A “recombinant” cell is a cell comprising a “recombinant” biomolecule. For example, a T cell that comprises a “recombinant” nucleic acid is a “recombinant” cell. A “recombinant microorganism” is a recombinant host cell that is a microorganism host cell. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell,” “recombinant cell,” and “host cell”, as used herein. A recombinant host cell may be an isolated cell or cell line grown in culture or may be a cell which resides in a living tissue or organism.

The term “polynucleotide”, “nucleic acid molecule”, “nucleic acid”, or “nucleic acid sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation. The nucleic acid (also referred to as polynucleotides) may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

A “synthetic” RNA, DNA or a mixed polymer is one created outside of a cell, for example one synthesized chemically.

The term “nucleic acid fragment” as used herein refers to a nucleic acid sequence that has a deletion, e.g., a 5′-terminal or 3′-terminal deletion compared to a full-length reference nucleotide sequence. In an embodiment, the nucleic acid fragment is a contiguous sequence in which the nucleotide sequence of the fragment is identical to the corresponding positions in the naturally-occurring sequence. In some embodiments, fragments are at least 10, 15, 20, or 25 nucleotides long, or at least 20, 30, 40, 50, 60, 70, 80, 90, 100, 1 10, 120, 130, 140, or 150 nucleotides long. In some embodiments a fragment of a nucleic acid sequence is a fragment of an open reading frame sequence. In some embodiments such a fragment encodes a polypeptide fragment (as defined herein) of the protein encoded by the open reading frame nucleotide sequence.

The nucleic acid can be purified. Preferably, the purified nucleic acid is more than 50%, 75%, 85%, 90%, 95%, 97%, 98%, or 99% pure. Within the context of this invention, a purified nucleic acid that is at least 50% pure means a purified nucleic acid sample containing less than 50% other nucleic acids. For example, a sample of a plasmid can be at least 99% pure if it contains less than 1% contaminating bacterial DNA.

The term “percent sequence identity” or “identical” in the context of nucleic acid sequences refers to the residues in the two sequences, which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32, and even more typically at least about 36 or more nucleotides. There are a number of different algorithms known in the art, which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

As used herein a “functional variant” or a given protein includes the wild-type version of said protein, a variant protein belonging to the same family, an homolog protein, or a truncated version, which preserves the functionality of the given protein. Typically the functional variant exhibit at least 70%, 75%, 80%, 85%, 90%, 95%, 97%, 98%, or 99% amino acid identity with the given protein.

As used herein, a “regulatory sequence” also named an “expression control sequence” refers to polynucleotide sequences which affect the expression of coding sequences to which they are operatively linked. Expression control sequences or regulatory sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” (also interchangeably named regulatory sequences) is intended to encompass, at a minimum, any component whose presence is essential for expression, and can also encompass an additional component whose presence is advantageous, for example, leader sequences and fusion partner sequences.

As used herein, “operatively linked” or “operably linked” to a linkage in which the expression control sequence (e.g.: regulatory sequences) is contiguous with the gene of interest to control its expression of the gene of interest. This term also include expression control sequences that act in trans or at a distance to control the expression of the gene of interest.

As used herein, the term “vector”, “transfer vector” “recombinant transfer vector”, or “gene transfer vector” is intended to mean a nucleic acid molecule capable of transporting a foreign nucleic acid (such as the polynucleotide or the nucleic acid encoding a hook fusion protein or the target fusion protein) to which it is linked.

One type of vector, which can be used in the present invention includes, in a non-limiting manner, a linear or circular DNA or RNA molecule consisting of chromosomal, non-chromosomal, synthetic or semi-synthetic nucleic acids, such as in particular a cosmid, artificial chromosomes such as a bacterial artificial chromosome (BAC) or a yeast artificial chromosome (YAC), a viral vector, a plasmid or an RNA vector. One of skill in the art would be well equipped to construct a vector through standard recombinant techniques, which are described in Sambrook et al. (1989) and Ausubel et al. (1994), both incorporated herein by reference.

Numerous vectors, into which a nucleic acid molecule can be inserted, in order to introduce it into and maintain it in a eukaryotic host cell including hematopoietic cell, are known per se; the choice of an appropriate vector depends on the use envisioned for this vector (for example, replication of the sequence of interest, expression of this sequence, maintaining of this sequence in extrachromosomal form, or else integration into the chromosomal material of the host), and also on the nature of the host cell.

A “plasmid,” generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Naked nucleic acid vectors such as plasmids are usually combined with a substance which allows them to cross the host cell membrane, such as a transporter, for instance a nanotransporter or a preparation of liposomes, or of cationic polymers. Alternatively, a naked nucleic acid may be introduced into said host cell using physical methods such as electroporation or microinjection. In addition, these methods can advantageously be combined, for example using electroporation combined with liposomes.

Another type of vector is a viral vector, wherein additional DNA segments may be ligated into the viral genome. Viral vectors are by nature capable of penetrating into cells and delivering polynucleotide(s) of interest into cells, according to a process named as viral transduction. Therefore, the polynucleotide sequences of interest are introduced into cells by contacting the recombinant viral vector with said cells. Viral vectors include retrovirus, adenovirus, adeno-associated virus (AAV), herpes virus, poxvirus, and other virus vectors. Retrovirus includes in particular type c retrovirus, human T cell leukemia virus (HTLV-1, HTLV-2) and lentivirus. Lentivirus includes in particular human immunodeficiency virus, including HIV type 1 (HIV1) and HIV type 2 (HIV2), feline immunodeficiency virus (FIV), bovine immunodeficiency virus (BIV), equine immunodeficiency virus (FIV), simian immunodeficiency virus (SIV), visna-maedi and caprine arthritis-encephalitis virus (CAEV).

Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome.

Moreover, certain vectors are capable of directing the expression of genes or nucleic acid sequences (i.e. encoding the hook fusion protein and/or the target fusion protein) to which they are operatively linked. Such vectors are referred to herein as “recombinant expression vectors” (or simply “expression vectors”). Expression vectors can contain a variety of “control sequences,” which refer to nucleic acid sequences necessary for the transcription and possibly translation of an operably linked coding sequence in a particular host organism.

As used herein, the term “mammal” refers to any member of the taxonomic class mammalia, including placental mammals and marsupial mammals. Thus, “mammal” includes humans, primates, livestock, and laboratory mammals. Exemplary mammals include a rodent, a mouse, a rat, a rabbit, a dog, a cat, a sheep, a horse, a goat, a llama, cattle, a primate, a pig, and any other mammal. In some embodiments, the mammal is at least one of a transgenic mammal, a genetically-engineered mammal, and a cloned mammal.

As used herein, “a hook protein” is usable in a system referred to as RUSH (retention using selective hooks) (see Boncompain et al., Nat. Methods 9:493-498, 2012, as well as WO2010142785 and WO201612623, which also describe the RUSH system). Typically the hook protein is a fusion protein, which allows the retention of a target protein containing a corresponding hook-binding domain in a donor compartment (i.e. the compartment from which the target protein originates) by a specific interaction with said target protein. When released from the interaction with the hook protein, the target protein is free to traffic toward its target compartment (i.e. the compartment to which the target protein is targeted). To control these two states, the specific interaction between the target protein and the hook is mediated by a reversible interaction between two interaction domains. In one embodiment, the interaction only occurs in the presence of a given ligand (“molecule-dependent” set-up, “MD”). In another embodiment, the interaction occurs by default and can be disrupted by a given ligand (“interaction-by-default” setup, “ID”). The removal or addition of the ligand acts like a switch to allow the synchronous release of the target protein from the donor compartment. When referring to a hook protein and a target fusion protein in a nucleic acid system, a vector system, an isolated cell, a kit or in a method or use of the invention, it is intended that the target fusion protein comprises a hook-binding domain, which corresponds to the hook domain of said hook fusion protein. Suitable hook domain/hook-binding domain couples are described below.

Chimeric Antigen Receptor

The invention encompasses a chimeric antigen receptor (CAR) comprising at least
an extracellular antigen-binding domain (binding domain),
the full DAP 10 protein, the full DAP 12 protein, or a functional variant thereof, and
a hook binding domain.

The CAR can contain one, two, three, or more of the binding domain and/or the hook-binding domain. The invention encompasses individually all possible combinations of the specific polypeptides and fragments thereof recited herein.

A binding domain according to the invention must be intended as an extracellular antigen-binding domain.

The invention comprises CARs containing a binding domain that comprises an antibody that binds specifically to a human polypeptide. The term “antibody” is meant to include polyclonal antibodies, monoclonal antibodies, fragments thereof, such as F(ab′)2 and Fab fragments, single-chain variable fragments (scFvs), single-domain antibody fragments (VHHs or Nanobodies, preferably camelid), and bivalent and trivalent antibody fragments (diabodies and triabodies).

Preferably, the antibody is a single-chain Fv antibody or a nanobody.

The antibody can be monospecific or multispecific for 2, 3, or 4 polypeptides. Preferably, the antibody is monospecific or bispecific.

Antibodies can be synthetic, monoclonal, or polyclonal and can be made by techniques well known in the art. Such antibodies specifically bind to human proteins via the antigen-binding sites of the antibody (as opposed to non-specific binding). Human proteins, polypeptide fragments, and peptides can be employed as immunogens in producing antibodies immunoreactive therewith. The human proteins, polypeptides, and peptides contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (C. A. Janeway, Jr. and P. Travers, Immuno Biology 3:9 (Garland Publishing Inc., 2nd ed. 1996)). Because folded proteins have complex surfaces, the number of epitopes available is quite numerous; however, due to the conformation of the protein and steric hindrance, the number of antibodies that actually bind to the epitopes is less than the number of available epitopes (C. A. Janeway, Jr. and P. Travers, Immuno Biology 2:14 (Garland Publishing Inc., 2nd ed. 1996)). Epitopes can be identified by any of the methods known in the art.

Thus, one aspect of the present invention relates to the antigenic epitopes of human proteins. Such epitopes are useful for raising antibodies, in particular monoclonal antibodies, as described in detail below.

Antibodies are defined to be specifically binding if they bind human proteins or polypeptides with a Ka of greater than or equal to about 10⁷ M⁻¹. Affinities of binding partners or antibodies can be readily determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice, or rats, using procedures that are well known in the art. In general, a purified human protein or polypeptide that is appropriately conjugated is administered to the host animal typically through parenteral injection. The immunogenicity can be enhanced through the use of an adjuvant, for example, Freund's complete or incomplete adjuvant. Following booster immunizations, small samples of serum are collected and tested for reactivity to human proteins or polypeptides. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKeam, and Bechtol (eds.), 1980. For example, the host animals, such as mice, can be injected intraperitoneally at least once and preferably at least twice at about 3 week intervals with isolated and purified human proteins or conjugated human polypeptides, for example a peptide comprising or consisting of the specific amino acids set forth above. Mouse sera are then assayed by conventional dot blot technique or antibody capture (ABC) to determine which animal is best to fuse. Approximately two to three weeks later, the mice are given an intravenous boost of the human protein or polypeptide. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as a labeled human protein or polypeptide, is added to each well followed by incubation. Positive wells can be subsequently detected. Positive clones can be grown in bulk culture and supernatants are subsequently purified over a Protein A column (Pharmacia). The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Antigen-binding fragments of such antibodies, which can be produced by conventional techniques, are also encompassed by the present invention. Examples of such fragments include, but are not limited to, Fab and F(ab′)2 fragments. Antibody fragments and derivatives produced by genetic engineering techniques are also provided.

The monoclonal antibodies of the present invention include chimeric antibodies, e.g., humanized versions of murine monoclonal antibodies. Such humanized antibodies can be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14: 139, May, 1993). Procedures to generate antibodies transgenically can be found in GB 2,272,440, U.S. Pat. Nos. 5,569,825 and 5,545,806.

Antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Publication No. WO 87/02671; Akira, et al. European Patent Application 0184187; Taniguchi, M., European Patent Application 0171496; Morrison et al. European Patent Application 0173494; Neuberger et al. PCT International Publication No. WO 86/01533; Cabilly et al. U.S. Pat. No. 4,816,567; Cabilly et al. European Patent Application 0125023; Better et al., Science 240: 1041 1043, 1988; Liu et al., PNAS 84:3439 3443, 1987; Liu et al., J. Immunol. 139:3521 3526, 1987; Sun et al. PNAS 84:214 218, 1987; Nishimura et al., Cane. Res. 47:999 1005, 1987; Wood et al., Nature 314:446 449, 1985; and Shaw et al., J. Natl. Cancer Inst. 80: 1553 1559, 1988); Morrison, S. L., Science 229: 1202 1207, 1985; Oi et al., BioTechniques 4:214, 1986; Winter U.S. Pat. No. 5,225,539; Jones et al., Nature 321:552 525, 1986; Verhoeyan et al., Science 239: 1534, 1988; and Beidler et al., J. Immunol. 141:4053 4060, 1988.

An immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9: 1370 1372; Hay et al. (1992) Hum Antibod Hybridomas 3:81 85; Huse et al. (1989) Science 246:1275 1281; Griffths et al. (1993) supra; Hawkins et al. (1992) J Mol Biol 226:889 896; Clackson et al. (1991) Nature 352:624 628; Gram et al. (1992) PNAS 89:3576 3580; Garrad et al. (1991) Bio/Technology 9: 1373 1377; Hoogenboom et al. (1991) Nuc Acid Res 19:4133 4137; and Barbas et al. (1991) PNAS 88:7978 7982. Once displayed on the surface of a display package (e.g., filamentous phage), the antibody library is screened to identify and isolate packages that express an antibody that binds a human protein or polypeptide. In a preferred embodiment, the primary screening of the library involves panning with an immobilized human protein or polypeptide and display packages expressing antibodies that bind immobilized human protein or polypeptide are selected.

In connection with synthetic and semi-synthetic antibodies, such terms are intended to cover but are not limited to antibody fragments, isotype switched antibodies, humanized antibodies (e.g., mouse-human, human-mouse), hybrids, antibodies having plural specificities, and fully synthetic antibody-like molecules.

The invention encompasses CARs comprising DAP 12, DAP10, or any functional variants thereof.

DAP10 and DAP12 are adapters that partner with most activating NKRs expressed in NK cells and all NKRs expressed in T cells (see Chen X, Bal F, Sokol L, et al. A critical role for DAP10 and DAP12 in CD8+ T cell-mediated tissue damage in large granular lymphocyte leukemia. Blood. 2009; 113(14):3226-3234).

In the immune system, DAP12 (DNAX-activation protein 12) is found in cells of the myeloid lineage, such as macrophages and granulocytes, where it associates, for instance, with the triggering receptor expressed on myeloid cell members (TREM) and MDL1 (myeloid DAP12-associating lectin 1/CLEC5A), both involved in inflammatory responses against pathogens like viruses and bacteria (for review, see Bakker A. B. et al., 1999. “Myeloid DAP12-associating lectin (MDL)-1 is a cell surface receptor involved in the activation of myeloid cells”. Proc. Natl. Acad. Sci. USA 96: 9792-9796). DAP12 possesses a single cytoplasmic immunoreceptor tyrosine-based activation motif (ITAM; D/ExxYxxL/Ix6-12YxxL/I) and signals by activating Syk protein tyrosine kinase, phosphoinositide 3-kinase (PI3K), and extracellular signal-regulated kinase (ERK/MAPK). This signaling pathway results in granule mobilization, target cell lysis, and cytokine production.

The DAP12 protein (Ref SeqGene: NG_009304.1, Uniprot ref: 043914) comprises a minimal extracellular region, mainly consisting of a cysteine residue that permits the creation of disulfide-bonded homodimers of DAP12, and which have no ligand-binding capacity. Intracellularly, DAP12 has a single ITAM, which after tyrosine phosphorylation recruits and activates notably Syk and ZAP70 in NK cells

By DAP12 it is herein intended to mean the wild-type human protein, one of its wild-type orthologs or a functional variant thereof. In any case, the functional variant comprises at least an extracellular domain, a transmembrane domain and an intracellular domain. Furthermore, a functional variant of DAP12 according to the invention also comprises at least the ITAM (immunoreceptor tyrosine-based activation motif) sequence. Preferably the human wild-type DAP12 protein is used. In a well-suited embodiment, the DAP12 signal peptide (corresponding to the first 21 amino terminal amino acids including the methionine) may be replaced by another signal peptide such as the CD8 signal peptide).

DAP10 (DNAX-activation protein 10) is a type I membrane protein of 93 amino acids (Gene bank ref: human DAP10 protein: AAD47911.1). It contains a short extracellular domain, a transmembrane domain and a short cytoplasmic domain. The DAP10 cytoplasmic domain comprises an YINM signaling motif which provides co-stimulatory signaling in conjunction with the ITAM-based TCR/CD3 complex in T cells.

By DAP10 it is herein intended to mean the wild-type human protein, one of its wild-type orthologs or a functional variant thereof. In any case, the functional variant comprises at least an extracellular domain, a transmembrane domain and an intracellular domain. Furthermore, a functional variant of DAP10 according to the invention also comprises at least the YxxM motif. Preferably, the human wild-type DAP10 protein is used. In a well-suited embodiment, the DAP10 signal peptide (corresponding to the first 21 amino terminal amino acids including the methionine) may be replaced by another signal peptide such as the CD8 signal peptide).

The inventors herein proposed that signal adaptor molecule DAP10 from the same family of DAP12, should be good candidates for CAR development. Indeed it has been shown that DAP10 triggers NK effector functions against malignant cells (Billadeau, Upshaw et al. 2003, Quatrini, Molfetta et al. 2015).

By full DAP10 or DAP12 it is preferably intended the human DAP10 or 12 according to the included database references and including or not the signal peptide as mentioned above.

Typically, DAP10 corresponds to the sequence SEQ ID NO: 14 (without including signal peptide), and DAP12 corresponds to the sequence SEQ ID NO: 15 (without including signal peptide).

Preferably the extracellular domain of the DAP10, DAP12, or of one of their functional variants is fused to the binding domain as previously defined. Typically said extracellular domain of the DAP10, DAP12, or of one of their functional variants is fused to an antibody such as a single-chain Fv antibody or a nanobody. In one alternative embodiment, said extracellular domain of the DAP10, DAP12, or of one of their functional variants is fused to a hinge fused to the binding domain. A hinge may be any linker amino acid sequence comprising 2 to 50 amino acids, such as a CD8 hinge

A CAR according to the invention may further encompass one or more additional activation domains selected from CD3-ζ chain (also shortly named) and the cytoplasmic domain of a costimulatory receptor such as CD28, 4-1 BB (CD137), OX40 (CD134), LAG3, TRIM, HVEM, ICOS, CD27, or CD40L. For example, a CAR according to the invention further comprises at least CD3-ζ. A CAR of the invention may also comprise CD3-ζ and at least one further activation domain selected from the above list. For example, a CAR of the invention can further comprise CD3-ζ and CD27.

Typically, a CAR according to the invention can comprise DAP 10 and further comprises a CD3-ζ chain activation domain. In particular, DAP-CD3-ζ chain is represented by SEQ ID NO:16.

Preferably, the CAR comprises additional activation domain(s) comprising a fragment of at least 50, 60, 70, 80, 90, 100, 1 10, 120, 150, or 200 amino acids of at least one additional activation domain selected from CD3-ζ chain (also shortly named ζ) and the cytoplasmic domain of a costimulatory receptors CD28, 4-1 BB (CD137), OX40 (CD134), LAG3, TRIM, HVEM, ICOS, CD27, or CD40L. In various embodiments, the CAR comprises additional activation domain(s) comprising a fragment of at least 20, 30, 40, 50, 60, 70, 80, 90, 100, 1 10, 120, 150, or 200 amino acids that shares at least than 90%, preferably more than 95%, more preferably more than 99% identity with the amino acid sequence of the additional activation domain above mentioned.

In a preferred embodiment of the invention, the CAR only comprises DAP10, DAP12 or a variant thereof in its intracellular domain.

The CAR can be purified. Preferably, the purified CAR is more than 50%, 75%, 85%, 90%, 95%, 97%, 98%, or 99% pure. Within the context of this invention, a purified CAR that is more than 50% (etc.) pure means a purified CAR sample containing less than 50% (etc.) other proteins. For example, a sample of a recombinant CAR purified from a host cell can be 99% pure if it contains less than 1% contaminating host cell proteins.

The invention encompasses CARs comprising a hook-binding domain. A “hook-binding domain” is a domain that reversibly binds directly or indirectly to the hook domain of a hook protein inside of the cell, and which binding leads to the retention of the target protein in the ER under appropriate conditions.

Suitable couple usable as hook-binding domain/hook domain (and the reverse) can be selected from Ftsz/ZipA, HPV E1/E2, recombinant antibody/epitope, recombinant epitope/hapten, proteinA/IgG domain, Fos/Jun. Interaction domain couples for which a molecule (ligand L) inhibiting the interaction is already known are preferred.

FtsZ and ZipA are bacterial proteins, which form part of the septal ring which forms during the replication of certain Gram-negative bacteria. Their interaction can be disrupted by addition of a small molecule named “compound 1” as a ligand L (see Wells et al. 2007 for review). Compound 1 (Wyeth Research (NY, USA)) can be used at concentrations ranging between 10 and 100 μM.

Streptavidin is a bacterial protein that binds with very high affinity to vitamin D-biotin. In vitro selection approaches have led to the discovery of synthetic peptides (streptavidin binding peptides, SBPs) that bind to Streptavidin and that can be competed out by biotin or biotin mimetic molecules from the ALiS (Artificial ligands of streptavidin) series (these compound are described in Terai T, Kohno M, Boncompain G, Sugiyama S, Saito N, Fujikake R, Ueno T, Komatsu T, Hanaoka K, Okabe T, Urano Y, Perez F, Nagano T. “Artificial Ligands of Streptavidin (ALiS): Discovery, Characterization, and Application for Reversible Control of Intracellular Protein Transport”. J Am Chem Soc. 2015 Aug. 26; 137(33):10464-7 and in Tachibana R, Terai T, Boncompain G, Sugiyama S, Saito N, Perez F, Urano Y. “Improving the Solubility of Artificial Ligands of Streptavidin to Enable More Practical Reversible Switching of Protein Localization in Cells”. Chembiochem: a European journal of chemical biology. 2017 Feb. 16; 18(4):358-62).

In a preferred embodiment, the hook-binding domain comprises a streptavidin-binding peptide (SBP), which can bind to a hook protein that bears a streptavidin hook domain. Biotin causes the release of the CAR containing the hook-binding domain from the hook by out-competing the SBP. The CAR is therefore free to move to the cell membrane.

Preferably, a system referred to as RUSH (retention using selective hooks) system can be employed, Boncompain et al., Nat. Methods 9:493-498, 2012, the content of which is hereby incorporated by reference (see also WO2010/142785).

Preferably, the hook-binding domain comprises the following SBP amino acid sequence:

(SEQ ID NO: 1)
MDEKTTGWRGGHVVEGLAGELEQLRARLEHHPQGQREP,

or is encoded by the nucleic acid sequence:

(SEQ ID NO: 2)
ATGGACGAGAAAACCACCGGCTGGCGGGGAGGCCACGTGGTGGAAGGACT
GGCCGGCGAGCTGGAACAGCTGCGGGCCAGACTGGAACACCACCCCCAGG
GCCAGAGAGAGCCC.

Shorter SBP fragments, deleted at their N-terminus and C-terminus may be used with identical efficacy. See Barrette-Ng, I. H., S. C. Wu, W. M. Tjia, S. L. Wong, and K. K. Ng. 2013, The structure of the SBP-Tag-streptavidin complex reveals a novel helical scaffold bridging binding pockets on separate subunits, Acta crystallographies. Section D, Biological crystallography 69:879-887.

Well-suited short SBP (sSBP) versions include the following sequences:

(SEQ ID NO: 3)
GHVVEGLAGELEQLRARLEHHPQGQREP
and
(SEQ ID NO: 9)
GGHVVEGLAGELEQLRARLEHHPQGQREP

The hook-binding domain is typically located in the intracellular domain and fused to the intracellular domain of DAP10, DAP12 or to a functional variant thereof. When the CAR comprises further activation domain(s), the hook-binding domain may be located in other positions, i.e., between the different co-stimulation elements.

In some embodiments the signal peptide of DAP10 or DAP12 may be replaced by another signal peptide. For example it has been noticed that the replacement of the signal peptide of DAP10 or DAP12 with the CD8 improves the CAR expression.

The following sequences provide example CAR sequences according to the invention. Of course these sequences are only illustrative and should not be intended as limitative. Typically the binding domain which is scFv CD19 may be replaced with any other binding domain and notably any other antibody. These examples also include DAP10 and DAP12 sequence wherein the signal peptide (consisting in the 21 N terminal amino acids including the methionine) have ben replace with the CD8 signal peptide. These examples also include the embodiment wherein the CAR further comprises an activation domain, in particular the CD3-ζ chain.

scFv CD19-myc-(CD8 signal peptide) DAP12-sSBP

(SEQ ID NO: 7)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSPAGEQKLISEEDLGRPLRPVQAQAQSDCSCSTVSPGV
LAGIVMGDLVLTVLIALAVYFLGRLVPRGRGAAEAATRKQRITETESPYQ
ELQGQRSDVYSDLNTQRPYYKHVVEGLAGELEQLRARLEHHPQGQREP

scFv CD19-myc-(CD8 signal peptide) DAP10-SBPdel-

(SEQ ID NO: 8)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSPAGEQKLISEEDLGRPQTTPGERSSLPAFYPGTSGSC
SGCGSLSLPLLAGLVAADAVASLLIVGAVFLCARPRRSPAQEDGKVYINM
PGRGHVVEGLAGELEQLRARLEHHPQGQREP

scFv aCD19-DAP10CD3-SBP

(SEQ ID NO: 13)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSPAGEQKLISEEDLGRPQTTPGERSSLPAFYPGTSGSC
SGCGSLSLPLLAGLVAADAVASLLIVGAVFLCARPRRSPAQEDGKVYINM
PGRGLKRVKFSRSADAPAYQQGQNQLYNELNLGRREEYDVLDKRRGRDPE
MGGKPQRRKNPQEGLYNELQKDKMAEAYSEIGMKGERRRGKGHDGLYQGL
STATKDTYDALHMQALPPRTGGHVVEGLAGELEQLRARLEHHPQGQREP

Nucleic Acids:

The invention encompasses a nucleic acid system comprising one or more nucleic acid(s), wherein said nucleic acid system comprises at least (a) a nucleic acid sequence encoding a CAR as previously defined.

In a preferred embodiment the CAR comprises only DAP10 or DAP12 as activation domain. Typically the full DAP10 or DAP12 sequence is used.

In various embodiments of the present invention, the nucleic acid system further comprises (b) a nucleic acid sequence encoding a target fusion protein comprising a hook-binding domain.

The nucleic acid(s) of the invention can be single-stranded or double-stranded. The nucleic acid can be an RNA or DNA molecule. Preferred nucleic acids encode an amino acid sequence of at least one of the SEQ ID NOs detailed herein. The invention also encompasses isolated nucleic acid(s) of the invention inserted into a vector.

In a nucleic acid system of the present invention nucleic acids sequences (a) and (b) can be included on the same nucleic acid or be separate nucleic acid molecules.

In one embodiment, a nucleic acid of the present invention comprises a nucleic acid sequence (a) encoding a CAR as previously defined and at least one nucleic acid sequence (b) encoding a hook protein.

A hook protein is a protein that prevents a CAR containing a hook-binding domain from exiting the endoplasmic reticulum (ER) or Golgi by reversibly binding, directly or indirectly, the hook-binding domain within the CAR. The retention can take place in the lumen of the ER or at its cytoplasmic face, depending on the design of the protein and the orientation of tagging with the interaction domains. Boncompain et al., Current Protocols in Cell Biology 15.19.1-15.19.16, December 2012, which is hereby incorporated by reference. Typically, the hook protein comprises a hook domain and an ER or Golgi retention domain. Preferably, the hook protein further comprises a transmembrane domain.

In some embodiments, the hook protein comprises an ER retention domain, such as a mutant of stromal interaction molecule 1 (STIM1-NN; a type I protein) that localizes in the ER but that cannot bind microtubules, an isoform of the human invariant chain of the major histocompatibility complex (li; a type II protein) that has an N-terminal arginine-based motif; or a C-terminal ER retention signal (Lys-Asp-Glu-Leu; KDEL). Boncompain et al., Nat. Methods 9:493-498, 2012, which is hereby incorporated by reference.

In an alternative embodiment, the retention domain can be a Golgi retention sequence such as Golgin-84.

Typically, the retention domain is an ER retention domain such the isoform of the human invariant chain of the major histocompatibility complex (li type II protein).

The retention domain can be fused to a hook domain in their luminal or cytoplasmic domain depending on the design of the CAR. Preferably, the hook domain is a cytosolic domain.

The hook domain binds to the hook-binding domain of the CAR. Suitable hook domain/hook-binding domain couples have been described in the previous section. Preferably the hook domain comprises a streptavidin that binds an SBP in the CAR.

Typically the hook protein is a transmembrane protein which comprises a cytosolic hook domain and an ER retention domain.

Preferably, the hook comprises a Streptavidin protein sequence, most preferably core Streptavidin. U.S. Pat. No. 5,672,691, which is hereby incorporated by reference.

Streptavidin protein sequences suitable to the present invention typically encompass the Streptavidin protein sequences as described below.

(SEQ ID NO: 4, wt streptavidin sequence)
MDPSKDSKAQVSAAEAGITGTWYNQLGSTFIVTAGADGALTGTYESAVGN
AESRYVLTGRYDSAPATDGSGTALGWTVAWKNNYRNAHSATTWSGQYVGG
AEARINTQWLLTSGTTEAN AWKSTLVG H DTFTKVKPSAAS I DAAK
KAGVN NG N PLDAVQQ

Suitable hook domains can also be selected from low affinity streptavidin mutant sequences. Such streptavidin mutant sequences can bind reversibly to biotin while keeping a high affinity for the streptavidin-binding protein (SBP). Accordingly, streptavidin protein sequences suitable for use in the present invention also encompass streptavidin sequences as described in Wu et al., PLoS ONE 8(7): e69530 (2013) and WO2013/038272 U.S. Pat. No. 9,353,161B2, which are hereby incorporated by reference. In particular, streptavidin sequences wherein the glycine at aa 49 (including the first methionine amino acid, or amino acid 48 if excluding said first methionine) of SEQ ID NO:1 or SEQ ID NO: 2 is replaced with a bulkier residue (e.g., threonine) to reduce the biotin binding affinity without affecting the SBP binding affinity are encompassed. Another mutation can also be introduced to further favor SBP binding over biotin (mutation S27A).

In particular, the skilled person in the art can create a single mutant containing a single mutation of serine to alanine substitution at residue 27, and a double mutant containing this change as well as a glycine to threonine substitution at residue 49 corresponding to full-length wild-type streptavidin (SEQ ID NO: 4). Although threonine is exemplified as a replacement residue for glycine 48, other residues with bulky side chains and high propensity for turns (Pt>0.83) are contemplated (e.g., Asp, Glu, Asn, Gin).

A monomeric core Streptavidin has also been constructed by Wu and Wong (2005) (see U.S. Pat. No. 7,265,205 B2 and SEQ ID NO:5 below).

(SEQ ID NO: 5)
MDPSKDSKAQVSAAEAGITGTWYNQLGSTFIVTAGADGALTGTYESAVGN
AESRYTLTGRYDSAPATDGSGTALGWRVAWKNNYRNAHSATTWSGQYVGG
AEARINTQWTLTSGTTEANAWKSTLRGHDTFTKVKPSAASIDAAKKAGVN
N GNPLDAVQQ.

As used herein, “Streptavidin” can refer to all forms of streptavidin (tetramer, core or monomer). In a preferred embodiment, a streptavidin sequence comprises the amino acid sequence as set forth in any of SEQ ID NO:4-5 as well as the low affinity variants as described above, or a variant thereof having at least 80% identity with SEQ ID NO:4 or SEQ ID NO:5, preferably 85%, 90, 95, 96, 97, 98, 99, 99.5% identity with such sequences. “Streptavidin” can also encompass Streptavidin homologs from other species, such as avidin or rhizavidin. Mutant of these natural biotin-binding proteins may also be used.

An example of a well-suited hook protein is the following (streptavidin fuse with II for cytoplasmic retention):

(SEQ ID NO: 6)
MHRRRSRSCREDQKPVTGDPSKDSKAQVSAAEAGITGTWYNQLGSTFIVT
AGADGALTGTYESAVGNAESRYVLTGRYDSAPATDGSGTALGWTVAWKNN
YRNAHSATTWSGQYVGGAEARINTQWLLTSGTTEANAWKSTLVGHDTFTK
VKPSAASIDAAKKAGVNNGNPLDAVQQVDYPYDVPDYAVGPMDDQRDLIS
NNEQLPMLGRRPGAPESKCSRGALYTGFSILVTLLLAGQATTAYFLYQQQ
GRLDKLTVTSQNLQLENLRMKLPKPPKPVSKMRMATPLLMQALPMGALPQ
GPMQNATKYGNMTEDHVMHLLQNADPLKVYPPLKGSFPENLRHLKNTMET
IDWKVFESWMHHWLLFEMSRHSLEQKPTDAPPKESLELEDPSSGLGVTKQ
DLGPVPM.

In some embodiments, the nucleic acid comprises a nucleic acid sequence encoding a hook protein, which is operably-linked to a promoter, for example but not limited to UBC or β2M, or any viral promoter, and a CAR comprising a hook-binding protein, operably-linked to an IRES or a 2A peptide. In a preferred embodiment, the hook is a streptavidin protein, preferably core Streptavidin, and the hook-binding protein is a streptavidin-binding protein.

The following sequences provide examples of CAR sequences according to the invention:
scFvCD19-myc-DAP10-SBPdel SEQ ID NO:10
scFvCD19-myc-DAP12-sSBP SEQ ID NO:11
scFv aCD19-DAP10CD3-SBP SEQ ID NO:13.
   Of course these sequences are only illustrative and should not be intended as limitative.

Vectors:

A vector system comprising one or more vector comprising:
(a) a nucleic acid comprising a nucleic acid sequence encoding a chimeric antigen receptor as previously defined, and optionally
(b) a nucleic acid encoding a hook protein as previously described
wherein the nucleic acids (a) and (b) are located on the same or on separated vectors.

Preferred nucleic acids (a) have been described in the prior section.

Preferably the hook-binding domain of the CAR comprises an SBP amino acid sequence and the hook domain of the hook protein comprise a streptavidin sequence. Preferred streptavidin sequence for the hook domain according to the invention have been described previously.

When the vector system comprises more than one vector, typically two or more vectors, said vectors are typically of the same type (e.g.: a lentiviral vector). In the following sections the vector can also be intended as “the one or more vector” or “the vector system”. Preferably the present invention encompasses a lentiviral vector system and notably a lentiviral particle system.

According to the invention, the vector can be an expression vector. The vector can be a plasmid vector.

A vector according to the invention is preferably a vector suitable for stable gene transfer and long-term gene expression into mammalian cells, such as by replication of the sequence of interest, expression of this sequence, maintaining of this sequence in extrachromosomal form, or else integration into the chromosomal material of the host. The recombinant vectors are constructed using standard recombinant DNA technology techniques and produced using conventional methods that are known in the art.

In some embodiments, a vector of the invention is an integrating vector, such as an integrating viral vector, such as in particular a retrovirus or AAV vector. Preferably, the viral vector is a lentiviral vector, most preferably an integrating viral vector.

Within the context of this invention, a “lentiviral vector” means a non-replicating non-pathogenic virus engineered for the delivery of genetic material into cells, and requiring lentiviral proteins (e.g., Gag, Pol, and/or Env) that are provided in trans. Indeed, the lentiviral vector lacks expression of functional Gag, Pol, and Env proteins. The lentivirus vector is advantageously a self-inactivating vector (SIN vector). The lentiviral vector comprises advantageously a central polypurine tract/DNA FLAP sequence (cPPT-FLAP), and/or insulator sequence (s) such as chicken beta-globin insulator sequence(s) to improve expression of the gene(s) of interest. The lentiviral vector is advantageously pseudotyped with another envelope protein, preferably another viral envelope protein, preferably the vesicular stomatis virus (VSV) glycoprotein. In some preferred embodiments, said lentiviral vector is a human immunodeficiency virus (HIV) vector.

Lentiviral vectors derive from lentiviruses, in particular human immunodeficiency virus (HIV-1 or HIV-2), simian immunodeficiency virus (SIV), equine infectious encephalitis virus (EIAV), caprine arthritis encephalitis virus (CAEV), bovine immunodeficiency virus (BIV) and feline immunodeficiency virus (FIV), which are modified to remove genetic determinants involved in pathogenicity and introduce new determinants useful for obtaining therapeutic effects.

The lentiviral vector may be present in the form of an RNA or DNA molecule, depending on the stage of production or development of said retroviral vectors. The lentiviral vector can be in the form of a recombinant DNA molecule, such as a plasmid, or in the form of a lentiviral vector particle (interchangeably named lentiviral particle in the context of the present invention), such as an RNA molecule(s) within a complex of lentiviral and other proteins.

Such vectors are based on the separation of the cis- and trans-acting sequences. In order to generate replication-defective vectors, the trans-acting sequences (e.g., gag, pol, tat, rev, and env genes) can be deleted and replaced by an expression cassette encoding a transgene.

Efficient integration and replication in non-dividing cells generally requires the presence of two c/s-acting sequences at the center of the lentiviral genome, the central polypurine tract (cPPT) and the central termination sequence (CTS). These lead to the formation of a triple-stranded DNA structure called the central DNA “flap”, which acts as a signal for uncoating of the pre-integration complex at the nuclear pore and efficient importation of the expression cassette into the nucleus of non-dividing cells, such as dendritic cells. In one embodiment, the invention encompasses a lentiviral vector comprising a central polypurine tract and central termination sequence referred to as cPPT/CTS sequence as described, in particular, in the European patent application EP 2 169 073.

Further sequences are usually present in cis, such as the long terminal repeats (LTRs) that are involved in integration of the vector proviral DNA sequence into a host cell genome. Vectors may be obtained by mutating the LTR sequences, for instance, in domain U3 of said LTR (AU3) (Miyoshi H et al, 1998, J Virol. 72(10):8150-7; Zufferey et al., 1998, J Virol. 72(12):9873-80). Preferably, the vector does not contain an enhancer. In one embodiment, the invention encompasses a lentiviral vector comprising LTR sequences, preferably with a mutated U3 region (AU3) removing promoter and enhancer sequences in the 3′ LTR.

The packaging sequence ψ (psi) can also be incorporated to help the encapsidation of the polynucleotide sequence into the vector particles (Kessler et al., 2007, Leukemia, 21 (9): 1859-74; Paschen et al., 2004, Cancer Immunol Immunother 12(6): 196-203). In one embodiment, the invention encompasses a lentiviral vector comprising a lentiviral packaging sequence ψ (psi).

Further additional functional sequences, such as a transport RNA-binding site or primer binding site (PBS) or a Woodchuck PostTranscriptional Regulatory Element (WPRE), can also be advantageously included in the lentiviral vector polynucleotide sequence of the present invention, to obtain a more stable expression of the transgene in vivo. can also be advantageously included in the lentiviral vector polynucleotide sequence of the present invention, to obtain a more stable expression of the transgene in vivo. In one embodiment, the invention encompasses a lentiviral vector comprising a PBS. In one embodiment, the invention encompasses a lentiviral vector comprising a WPRE and/or an IRES.

Thus, in a preferred embodiment, the lentiviral vector comprises at least one cPPT/CTS sequence, one ψ sequence, one (preferably 2) LTR sequence, and an expression cassette including a transgene under the transcriptional control of a β2ηη or class I MEW promoter.

Preferably, a vector (i.e. a recombinant transfer vector) of the invention is an expression vector comprising appropriate means for expression of the hook fusion protein and/or the target fusion protein in a host cell.

Various promoters may be used to drive high expression of the nucleic acid sequence encoding the hook fusion protein and/or the target fusion protein. The promoter may be a tissue-specific, ubiquitous, constitutive or inducible promoter. Preferred promoters are notably functional in T cells and/or NK cells, preferably human T cells and human NK cells. In particular, preferred promoters are able to drive high expression of the hook fusion protein and the target fusion protein (notably a CAR as previously defined) from lentivectors in T cells or NK cells, preferably human T cells or NK T cells. For example, a promoter according to the invention can be selected from phosphoglycerate kinase promoter (PGK), spleen focus-forming virus (SFFV) promoters, elongation factor-1 alpha (EF-1 alpha) promoter including the short form of said promoter (EFS), viral promoters such as cytomegalovirus (CMV) immediate early enhancer and promoter, retroviral 5′ and 3′ LTR promoters including hybrid LTR promoters, human ubiquitin promoter, MHC class I promoter, MHC class II promoter, and β2 microglobulin (β2ηη) promoter. The promoters are advantageously human promoters, i.e., promoters from human cells or human viruses such as spleen focus-forming virus (SFFV).

Human ubiquitin promoter, MHC class I promoter, MEW class II promoter, and β2 microglobulin (β2ηη) promoter are more particular preferred. Preferably, the MHC class I promoter is an HLA-A2 promoter, an HLA-B7 promoter, an HLA-Cw5 promoter, an HLA-F, or an HLA-E promoter. In some embodiments the promoter is not a CMV promoter/enhancer, or is not a dectin-2 or MHCII promoter. Such promoters are well-known in the art and their sequences are available in sequence data base.

Typically, lentiviral particles refer to the extracellular infectious form of a virus composed of genetic material made from either DNA or RNA (most preferably single stranded RNA) surrounded by a protein coat, called the capsid, and in some cases an envelope of lipids that surrounds the capsid. Thus a lentiviral vector particle (or a lentiviral particle) comprises a lentiviral vector as previously defined in association with viral proteins. The vector is preferably an integrating vector.

The RNA sequences of the lentiviral particle can be obtained by transcription from a double-stranded DNA sequence inserted into a host cell genome (proviral vector DNA) or can be obtained from the transient expression of plasmid DNA (plasmid vector DNA) in a transformed host cell. Appropriate methods for designing and preparing lentiviral particles in particular for therapeutic application are well-known in the art and are for example described in Merten O W, Hebben M, Bovolenta C. Production of lentiviral vectors. Mol Ther Methods Clin Dev. 2016 Apr. 13; 3:16017.

Preferably the lentiviral particles have the capacity for integration. As such, they contain a functional integrase protein. Non-integrating vector particles have one or more mutations that eliminate most or all of the integrating capacity of the lentiviral vector particles. For, example, a non-integrating vector particle can contain mutation(s) in the integrase encoded by the lentiviral pol gene that cause a reduction in integrating capacity. In contrast, an integrating vector particle comprises a functional integrase protein that does not contain any mutations that eliminate most or all of the integrating capacity of the lentiviral vector particles.

In one embodiment of the present invention, the nucleic acid encoding the CAR and hook protein are inserted into separate vectors.

In another embodiment, the nucleic acid encoding the CAR and hook protein are inserted into the same vector.

In the later embodiment, each coding sequence (i.e. the nucleic acids encoding respectively the hook protein and the CAR) can be inserted in a separate expression cassette. Each expression cassette therefore comprises the coding sequence (open reading frame or ORF) functionally linked to the regulatory sequences which allow the expression of the corresponding protein (hook fusion protein and target fusion protein) in the host cell, such as in particular promoter, promoter/enhancer, initiation codon (ATG), codon stop, transcription termination signal.

Alternatively, the hook fusion protein and the target fusion protein may also be expressed from a unique expression cassette using an Internal Ribosome Entry Site (IRE S), or a self-cleaving 2A peptide inserted between the two coding sequences to allow simultaneous expression.

Nucleic acids encoding the hook protein and the CAR can be inserted in a single expression vector, said single vector comprising a bicistronic expression cassette. Vectors containing biscitronic expression cassette are well known in the art. Advantageously, bicistronic expression cassettes contain an Internal Ribosome Entry Site (IRES) that enables the expression of both fusion proteins from a single promoter. Suitable commercially available bicistronic vectors can include, but are not limited to plasmids of the pIRES (Clontech), pBud (Invitrogen) and Vitality (Stratagene) series. Preferably, the nucleic acid located upstream of the IRES sequence is operably-linked to a promoter. Preferably the nucleic acid encoding the hook protein is inserted upstream of the IRES sequence and the nucleic acid encoding the target fusion protein is inserted downstream of said IRES sequence to ensure that enough the hook fusion protein will be sufficiently expressed to retain every target fusion protein. In some embodiments multicistronic expression vectors may be used wherein more than one, typically at least two, nucleic acids encoding each a distinct hook and at least one nucleic acid encoding a target fusion protein are inserted.

A self-cleaving 2A peptide can also be used in replacement of IRES. Such strategy is highly advantageous because of its small size and high cleavage and translation efficacy between nucleic acid sequences upstream and downstream of the 2A peptide. Suitable 2A peptide according to the invention are notably described in Kim J H, Lee S-R, Li L-H, et al. High Cleavage Efficiency of a 2A Peptide Derived from Porcine Teschovirus-1 in Human Cell Lines, Zebrafish and Mice. PLoS ONE. 2011; 6(4):e18556. 2A peptides can be selected from FMDV 2A (abbreviated herein as F2A); equine rhinitis A virus (ERAV) 2A (E2A); porcine teschovirus-1 2A (P2A) and Thoseaasigna virus 2A (T2A). P2A or T2A peptide is preferred. Although the use of a self-cleaving 2A peptide is generally recommended when a stoichiometric expression of the sequences located upstream and downstream of the 2A peptide, the inventors have found that it could still be used advantageously in the present “RUSH” context. Unexpectedly, although it was thought that the hook-fusion protein needed to be in excess of the CAR comprising an SBP sequence, the inventors have found that this is not the case.

Thus in one embodiment, the invention encompasses a vector notably and expression vector, most preferably a lentiviral vector, comprising a nucleic acid encoding the hook protein as previously defined which is inserted upstream of a 2A peptide sequence and a nucleic encoding the CAR which is inserted downstream of the 2A peptide.

The present invention also encompasses a viral particle system, wherein the one or more viral particle comprises a viral vector, typically an integrating viral vector, as previously defined. Preferably, the viral vector is a lentiviral vector and the viral particle is a lentiviral particle. In one embodiment, the viral particle system comprises separated particles comprising a viral vector encoding respectively the hook protein and the CAR. In an alternative embodiment, the viral particle system comprises one particle comprising viral vector encoding both the hook fusion protein and the CAR as previously described. The nucleic acid sequence encoding the hook protein and the nucleic acid sequence encoding the CAR are preferably expressed from a unique expression cassette as defined above.

Isolated Cells of the Invention

The invention encompasses isolated cells, particularly cells of the immune system, comprising vectors and notably a viral vector particle encoding at least a CAR as previously described. Preferably the vectors and/or lentiviral particles further comprise a nucleic acid sequence encoding a hook protein. Preferably, the cells are T cells or NK cells.

In one embodiment, the cell contains the vector and/or viral vector particle integrated into the cellular genome. In one embodiment, the cell contains the vector stably expressing the CAR and preferably also the hook protein. In one embodiment, the cell produces lentiviral vector particles encoding the CARs and preferably also the hook protein.

In various embodiments, the invention encompasses a cell line, a population of cells, or a cell culture comprising vectors, notably viral vector particles, encoding the CAR and preferably also the hook protein.

Kit According to the Invention:

The present invention also relates to a kit comprising a nucleic acid comprising at least a nucleic acid system as above defined and comprising at least a nucleic acid sequence encoding a CAR of the invention. Preferably said nucleic acid system further comprises a nucleic acid sequence encoding a hook protein. Preferentially, said nucleic sequences are comprised in the same nucleic acid.

The kit of the invention may alternatively comprise a vector system, a viral particle system, or a host cell as previously defined. Preferably, the kit comprises a vector encoding a CAR and its corresponding hook protein. Preferably the vector is a viral vector notably a lentiviral vector. In another advantageous embodiment, the kit comprises at least a viral vector particle comprising a viral vector according to the invention.

Preferably the hook protein comprises a streptavidin sequence and the CAR comprises an SBP sequence.

The kit further can comprises a specific ligand. When the hook protein comprises a streptavidin sequence and the CAR comprises an SBP sequence, the ligand can be selected from biotin or a biotin mimetic molecule selected from ALiS.

Methods for Expressing a CAR in a Cell:

The present invention encompasses methods for expressing a CAR in a cell. The method comprises transducing a cell with a lentiviral vector system or lentiviral particle vector of the invention under conditions that allow the expression of the CAR, and preferably expanding the T and NK cells.

The cells are preferably mammalian cells, particularly human cells. Particularly preferred are human non-dividing cells.

Preferably, the cell are immune cells, notably primary NK cells or T cells, and preferably expanded T cells T cells.

The method can further comprise harvesting or isolating the CAR.

The lentiviral vector or lentiviral particle vector preferably comprises a promoter of the invention.

In one embodiment, the method comprises treating the cells with biotin or a biotin mimetic molecule (ALis as mentioned in a previous section) to release the CAR from the hook. Preferably, the cells are treated with biotin at an initial concentration of, at least, 0.2, 0.4, 0.8. 1.6, 2.5, 5, 10, 20, 40, or 80 μM.

In one embodiment, the invention encompasses a method for expressing a CAR as previous defined, comprising inserting a promoter as described in a previous section into a lentiviral vector such that it direct the expression of a nucleic acid encoding a CAR and transducing a cell, preferably a T or NK cell, with the vector containing the promoter, and optionally, treating the cell with biotin at an initial concentration of, at least, 0.2, 0.4, 0.8. 1.6, 2.5, 5, 10, 20, 40, or 80 μM.

Medical Uses of the Invention:

The present invention further relates to a hook fusion protein, or a nucleic acid or a nucleic acid system, or a vector system or a viral particle or a host cell or a kit as herein described as a medicament, in particular for use in immunotherapy, most particularly for adjuvant immunotherapy. In particular, the present invention relates to the use of a vector system, notably a viral vector system and in particular a lentiviral vector system as a medicament. Said vector system comprises a nucleic acid sequence encoding a hook protein and a nucleic acid sequence encoding a CAR. Preferably also the hook protein has a hook domain comprising a streptavidin sequence and the CAR has a hook-binding domain comprising an SBP sequence.

As previously mentioned, the present invention provides new CAR which intracellular trafficking and cell membrane expression can be timely controlled. This innovation is of particular relevance as CAR exhibits by nature a high cytotoxicity.

The invention can also be used in treatment protocols against tumors and cancers and especially could be used in protocols for immunotherapy or vaccination therapy against cancers and tumors.

As previously mentioned the nucleic acid sequences as above mentioned can be included in separated vectors or included in the same vector. Preferably the vector is an integrating viral vector, most preferably an integrating lentiviral vector.

In a preferred embodiment, the invention relates to the viral vector as above mentioned or to a viral vector particle comprising said viral vector for use as a medicament. Said viral vector or viral vector particle can be used for example in a therapeutic composition or vaccines which are capable of inducing or contributing to the occurrence or improvement of an immunological reaction with the CAR encoded by the vector. The invention therefore also encompasses an immunogenic composition comprising a viral vector as previously defined.

The invention encompasses methods of administration of a viral vector (notably a lentiviral vector) to a human. Preferred modes of administration include reinfusion of the modified T or NK cells, preferably intravenously or intra-articular administration, most preferably intra-tumoral administration.

In one embodiment, viral vector particles according to the invention can be administered to T or NK cells. The obtained modified T cells or NK cells can be further administered to a human.

The viral vector and viral vector particles according to the invention have the ability to redirect the specificity and function of T lymphocytes and/or other immune cells such as NK cells. They can rapidly generate T cells targeted to a specific tumor antigen or an antigen relevant in other pathologies like auto-immune diseases.

The viral vector and viral vector particles of the invention can therefore be used in methods of treatment and methods of inducing an immune response comprising administering the viral vector to a cell, preferably a T or NK cell, administering the cell to a host, and generating a specific immune response that redirects the specificity and function of T lymphocytes and/or other immune cells.

A particular advantage of the immunogenic compositions of the invention is that they can be used to redirect the specificity and function of T lymphocytes and other immune cells against multiple antigens against which the CAR in the vector or vector particles are directed.

As a result, the invention encompasses a composition that could be used in therapeutic vaccination protocols. In particular, it can be used in combination with adjuvants, other immunogenic compositions, chemotherapy, or any other therapeutic treatment. Thus the present invention also relates to the chimeric antigen receptor, or the vector system or the viral vector particle system, or the kit as herein described for use for inducing an immune response in a human, in particular for inducing a controlled immune response in a human.

The method can further comprise administering biotin or a biotin mimetic (ALiS as previously described) to the human to release the target fusion protein and in particular the CAR from the ER Preferably, the biotin is administered at an initial concentration of at least, 0.2, 0.4, 0.8. 1.6, 3.2, 5, 10, 20, 40, or 80 μM.

Having thus described different embodiments of the present invention, it should be noted by those skilled in the art that the disclosures herein are exemplary only and that various other alternatives, adaptations, and modifications may be made within the scope of the present invention. Accordingly, the present invention is not limited to the specific embodiments as illustrated herein.

### Results

1—RUSH Control of DAP10/12 Based CAR Trafficking

Methods and Material:

Constructs

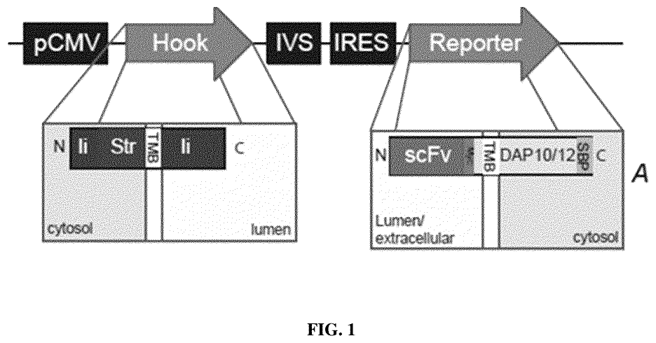

FIG. 1 shows a schematic representation of the CAR-based RUSH constructs.

The RUSH constructs simultaneously express a HOOK protein containing streptavidin (Str) for cellular retention/release of the cargo protein (i.e.: CAR fusion protein containing a hook-binding domain).

To this end, a biscistronic plasmid was used with an IRES (Internal Ribosome Entry Site) to allow simultaneous expression of hook (i.e.: the hook protein) and reporter (Boncompain and Perez 2012, Abraham, Gotliv et al. 2016).

The hooks have been previously described in Boncompain et al (2012). Those are inserted in the bicistronic vector using multicloning sites and the reporter using the typical cloning cassettes of the previously published RUSH vector. The hooks used present core streptavidin in the cytoplasmic face. More particularly, the Hook is a fusion protein between core streptavidin and an isoform of the human invariant chain of the major histocompatibility complex (Ii; type II protein) containing a N-terminal arginine based motif for ER retention (Boncompain and Perez 2012, Abraham, Gotliv et al. 2016).

The following reporters were designed:

DAP10 or DAP12 were fused in their extra-cellular to an scFv directed against CD19 and in their intra-cellular domain (Carboxy-terminal) to an SBP. For development purpose, a myc tag can be added close to the scFv in the extracellular domain. The reporters were built using gene syntheses (gBlocks Gene Fragments—Integrated DNA Technologies)

Cell Culture and Transfection:

HeLa cells were cultivated at 37° C. and 5% of CO₂ in Dulbecco's modified Eagle medium (DMEM) supplemented with 10% FBS (Biowest), 1 mM sodium Pyruvate and 100 μM of penicillin and streptomycin (Invitrogen). HeLa cells were transfected with the plasmid of interest using Calcium phosphate protocol in the presence of 25 mM of HEPES.

Briefly, the plasmids coding the sequence of CAR based RUSH (2.5 ug per 1 mL of final volume) were add to 1 mM tris-HCl pH 8.02 buffer followed by the addition of 10% of CaCl₂ and incubated for 5 min (RT). Then this mix was add drop by drop into 2× concentrate HEBS buffer (160 mM NaCl, 1.5 mM Na₂HPO₄, 50 mM Hepes PH 7.04-7.05) while vortexing. The cells were incubated with this solution overnight at 37° C. and 5% of CO₂.

Time Course Release from the ER Upon Biotin Addition:

The cells were seeded into a glass coversplips for fixed cell immunofluorescence and/or live imaging.

In the next day, the cells were transfected with the plasmids coding the construct of interest as above described. For the steady state of the protein/construct, 40 uM final concentration of biotin was add (4 mM stock solution) just after addition of the transfection solution. The presence of biotin will prevent the interaction of the reporter with the hook, allowing the normal traffic of the reporter.

In the next day, the cell in the coversplips were incubated at different time points with a final concentration of 40 μM of biotin, allowing the traffic of the reporter and then prepared for immunofluorescence.

Immunofluorescence:

Cells coated in the coversplips were washed once in 1×PBS buffer, fixed in 3% of paraformaldehyde (PFA) (10-15 min, RT), washed (2×) and incubated with 50 mM of NH₄Cl (5 min, RT) to quench free aldehydes. The cells were then permeabilized using a solution of PBS containing bovine serum Albumin (BSA, 0.5%, Sigma-Aldrich) and saponin (Sapo, 0.05% Sigma-Aldrich)(15 min, RT). When the protein was not fluorescent labelled, we used antibodies for their detection. These include the monoclonal anti human NKG2D (1/800, Biolegend), and anti-myc tag from mouse (1/2000, clone 9E10) or anti-myc from rabbit (1/500, Cell Signaling). The coverslip were mounted in Mowiol (Calbiochem) supplemented with DAPI (4′,6-Diamidino-2-phenylindole) for DNA staining.

Results

The RUSH system and other related systems were adapted to accommodate different CARs, namely DAP10/DAP12 (see the scheme of FIG. 1).

The DAP10 and DAP12 based CARs were constructed by fusing the scFv CD19 with Myc tag DAP10 and DAP12 followed by a classical or smaller streptavidin binding peptide (sSBP, with 28 amino-acids (aa), instead of the typical 36 aa)) (FIG. 1-A).

The presence of the SBP allows the retention of the CARs in the endoplasmic reticulum (ER) by a specific interaction with the Hook composed of streptavidin fused to the isoform of the human invariant chain of the major histocompatibility complex (Ii; a type II protein) bearing an ER retention arginine-based motif at the N-terminal.

This type of hook allows the cytosolic retention of the reporter in the ER (the K_{D} of SBP to Streptavidin interaction is around 10⁻⁹ M). Biotin competes with SBP-CAR for the binding to streptavidin. As biotin binds to the same site and has a higher affinity for the streptavidin (K_{D}=10⁻¹⁵ M), addition of biotin leads to the release of the SBP-tagged CAR. The CAR is then “free” to traffic to the cells surface.

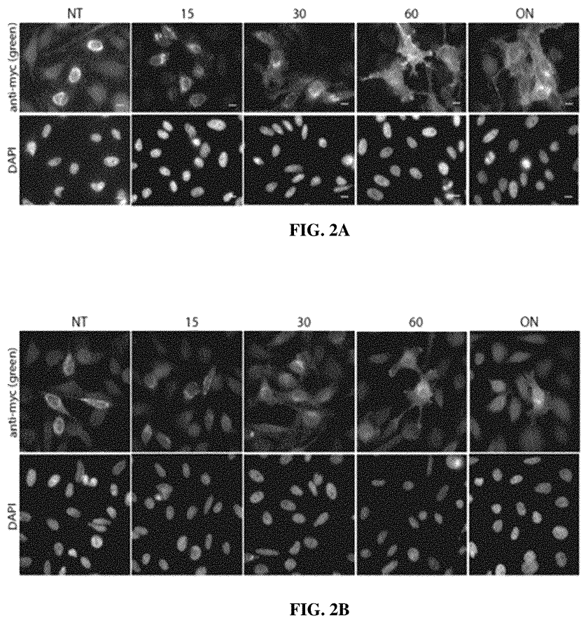

The CAR-RUSH constructs (K_{D}=10⁻⁹ M) were validated using transient transfection (calcium precipitation protocol) in HeLa cells (FIG. 2).

The results shows that the DAP 10/12-based CAR, scFv(CD19)-DAP10-sSBP and scFv(CD19)-DAP12-sSBP, are efficiently retained in the ER, and released upon addition of biotin (see FIG. 2).

At 15 min, the majority of the CARs reach the Golgi apparatus

At 30 min, cell membrane expression of CARs is observed although some fluorescence labelling remains in the Golgi apparatus (FIG. 2).

At 60 min, the majority of CARs is at the cell membrane (FIG. 2).

These results show that the RUSH system can be adapted efficiently to control the traffic of various types of CARs and in particular cell surface expression of CARs.

2—Lentiviral Vector Including DAP10/12 Based CARs have Improve Transduction Efficacy

Methods and Material:

Generation of Lentiviral Plasmid

The nucleic acid sequence for scFvCD19 CAR June (scFvCD19-myc-tmCD8-41BB-CD3z-SBP) were generated by gene synthesis by DC Biosciences. This plasmid was included downstream a 2A self-cleaving peptide (2A) from Thoseaasigna virus 2A (T2A)(Kim J H, Lee S-R, Li L-H, et al. High Cleavage Efficiency of a 2A Peptide Derived from Porcine Teschovirus-1 in Human Cell Lines, Zebrafish and Mice. Thiel V, ed. PLoS ONE. 2011; 6(4):e18556) and Y-FAST fused to Puromycin resistance gene.

The nucleic acid sequence for the CAR scFvCD19-myc-DAP10-SBP was also generated by gene synthesis by gBlocks Gene Fragments—Integrated DNA Technologies. This nucleic acid sequence was then amplified by PCR to insert restriction, PmeI and PacI sites, required to subclone into the recipient lentiviral vector.

The receiving lentiviral vector used was generated based on the vector from pTRIP-SFFV-mtagBFP-2A described in Gentili et al., Science 2015, by substitution of mtagBFP by the synthetized gene of scFvCD19 CAR June (scFvCD19-myc-tmCD8-41BB-CD3z-SBP)-2A-Yfast-Puromycin using restriction site PmeI/XhoI. Of note, the lentiviral vector pTRIP-SFFV-mtagBFP-2A was previously modified by removing a PacI restriction site present in its backbone using MUNG BEAN (NEB) protocol.

Production of Lentivirus

For the production of lentiviral particles, the expression plasmids psPAX containing Gag, Pol, Rev, and Tat genes required for the packaging and pMD.2 g for the expression of VSVG envelope plasmid was used. The lentiviral particles were produced by transient transfection of cell line HEK293FT cells using the polyethylenimine (PEI MAX, polyscience) protocol. The ratio of the plasmids used was, per T75 cm3 flask with 10×106 cells, 1.35 μg pMD.2 g, 3.24 μg of psPAX and 4.5 μg of the protein of interest for 12300 bp of plasmid. 48 hours after, the supernatant containing the lentiviral particles were filter using a 45 μm filer and kept at 4 C prior to transduction of the cells of interest.

Cell Transduction

HeLa cells were seeded at 10 000 cells/well in a 96 well plate the day before transduction. In the following day, the supernatant containing the lentiviral particles was diluted 50% in Dulbecco's modified Eagle's medium (DMEM) complete medium (10% fetal bovine serum (FBS); 2 mM L-glutamine; 100 units/mL penicillin G; 100 μg/mL streptomycin), i.e. 60 ml of lenti supernatant+60 ml of medium in a serial dilution of ½ in a 96 well plate. Then 50 μL of the serial dilution mix was transferred to the seed HeLa cells (in 50 μL of DMEM complete).

72 hours after the CAR expression was evaluated by flow cytometry using Yellow Fluorescence-Activating and absorption-Shifting Tag (Y-FAST) that is able to react with 4-hydroxy-3-methylbenzylidene-rhodanine (HMBR), activating their green fluorescence (Plamont et al, PNAS, 2016).

Briefly, the transduced cells were detached using 0.25% tripsin-EDTA (1×) phenol red, Gibco® solution and incubated during 5 min at 37 C. Then 150 μL of DMEM complete was added, and the mixture was centrifuged for 5 min, at 900 rpm. The ells were then transferred to a new 96 well plate, followed by two washes in PBS. The flow cytometry analysis was performed in the transduced cells re-suspended in PBS and minutes before acquisition, fluorogenic HMBR was added for Y-FAST reaction.

Results:

To compare the efficiency of cells transduction between (1) the lentiviral vector for scFvCD19 CAR June (scFvCD19-myc-tmCD8-41BB-CD3z-SBP) fused to 2A-Y-FAST: Puromycin and (2) scFvCD19-DAP10 CAR (scFvCD19-myc-DAP10-SBP) also fused to 2A-Y-FAST:Puromycin, we generated lentiviral particles by transient transfection of HEK293FT using the same base pair (bp) ratio of the two CARs, to be able to compare the efficiency of transduction.

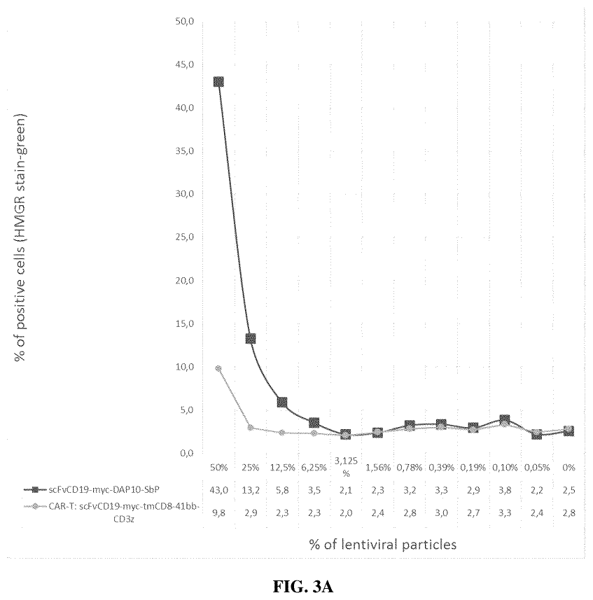

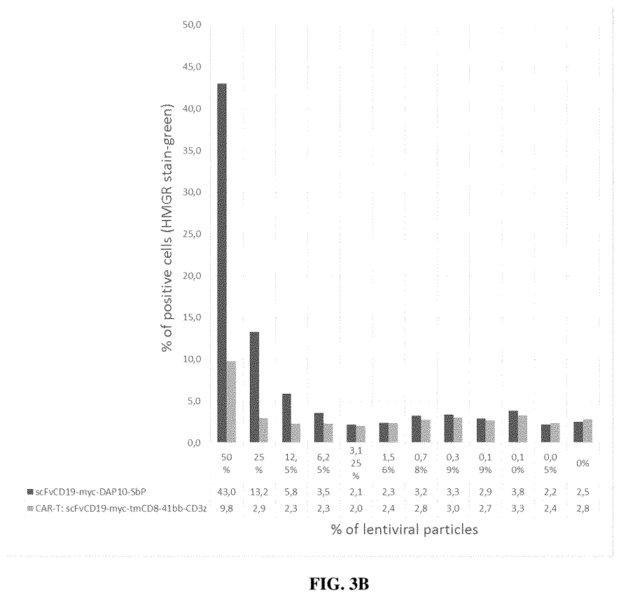

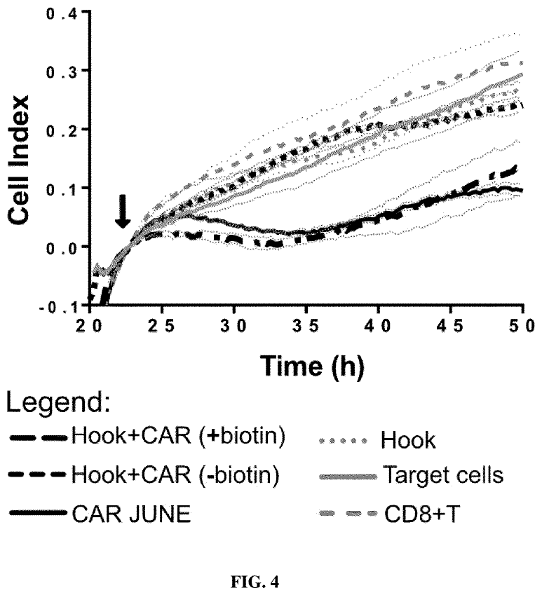

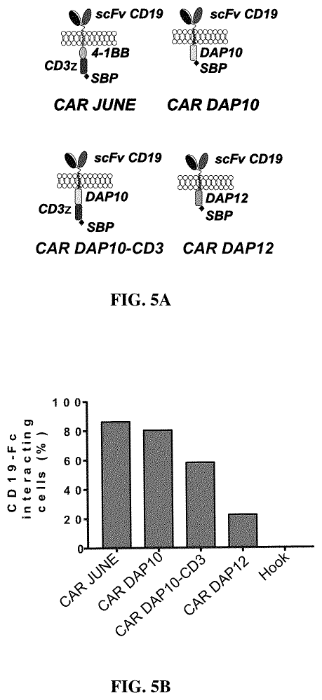

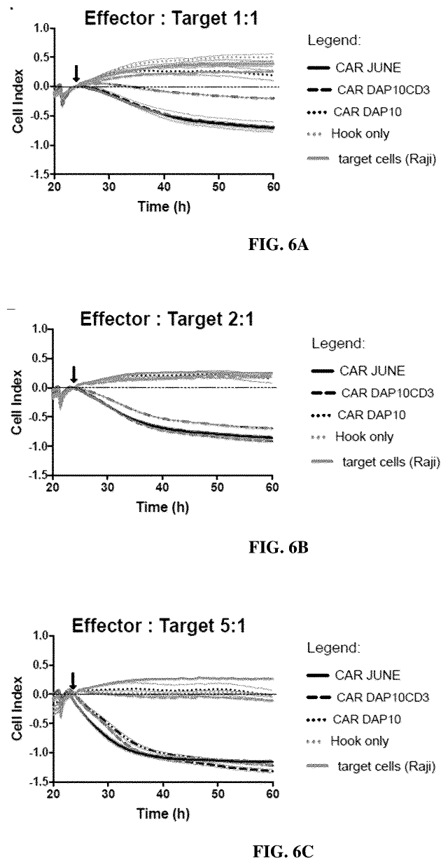

We observed a higher transduction efficiency (4×(˜40% vs 10%, scFvCD19-DAP10 CAR/scFvCD19 CAR June, respectively) of the 11.8 kbp scFvCD19-DAP10 CAR when compared with the 12.3 kbp scFvCD19 CAR June (FIG. 3).

These results show that the CAR design and in particular the size construct is highly relevant for transduction efficiency and thus for the expression of the CARs in immune cells.

As immune cells are known to be naturally difficult to transduced it is therefore of high importance to propose CARs which allow obtaining high transduction efficacy without compromising the activation efficiency of immune cells. The CAR as herein described are highly advantageous as their size is much smaller as compared to the typical CAR of the prior art.

3—Adaptation of CAR in T Cells to the RUSH Technology

Methods and Material:

Plasmid Construction

Lentiviral vector pTRIP-SFFV-tagBFP-P2A (Gentili et al., Science, 2015) kindly provided by Nicolas Manel was used for cloning the anti-CD19 CARs and Hook. Briefly, the mtagBFP-2A-CARs were constructed by PCR amplification of the synthetized CAR JUNE (scFvCD19-myc-(hinge&Tm) CD8-41BB-CD3-SBP), CAR DAP10 (scFvCD19-myc-DAP10-SBP), CAR DAP10-CD3 (scFvCD19-myc-DAP10-CD3ζ-SBP) and CAR-DAP12 (scFvCD19-myc-DAP10-SBP). These amplicons were cloned into a pTRIP-SFFV-tagBFP-2A plasmid using BamHI and Sall (NEB). To generate pTRIP-SFFV-CARJUNE-2a-YfastPuro, the DNA encoding CAR June was synthesized and cloned into the previously modified vector pTRIP-SFFV-2a-YfastPuro using PmeI and PacI (NEB). For the generation of pTRIP-SFFV-YfastPuro-2A-Str-Ii, the sequence of tagBFP-P2A with the cytosolic hook based on the human isoform of invariant chain of the major histocompatibility complex (Ii) fused with streptavidin (Str) (Boncompain et al., Nature Methods, 2012) was amplified by PCR and cloned into the previously modified pTRIP-SFFV-YastPuro-2A using AscI and SpeI. The mtagBFP was exchanged to Yfast (Plamont et al., PNAS, 2016) fused to puromycin by PCR amplification followed by cloning using BamHI and SacII.

Lentiviral Particles Production

The lentiviral particles were produced by transfection of HEK293 ft cells with psPAX2 plasmid and the pseudotype encoding VSVG (pMD2.G) kindly provided by Francois-Xavier Gobert, using polyethyleneimine (PEI) precipitation. After 48 h of incubation, the supernatant was collected and new medium was added to the cells to continue lentiviral production for more 24 h. The collected supernatant was filtered (0.20 μm-pore-size filter), and centrifuged at 31 000 g for 90 min at 4° C. on a 20% sucrose-PBS cushion. Pellets were resuspended in DC medium (complete medium supplemented with 10 mM HEPES and 0.5 mM (3-Mercaptoethanol), aliquoted and stored at −80° C. for several weeks to 2-3 months. Lentiviral particles titration was performed by infecting 10,000-15,000 HEK293 ft cells with serial dilutions of the lentiviral particles in a 96 well plate for 72 days at 37° C. The lentiviral titter (TU/mL) was estimated by flow cytometry using the mtagBFP fluorescent protein. The lentiviral titter (TU/mL) was calculated using the formula (P×N)/(V×D) with P been the number of positive cells for the fluorescent protein (for a maximum for 20% positive cells), N the number of cells per well, D the dilution factor of the viral particles and V the total volume in mL.

CD8+ T Cells Isolation and Transduction

Normal donor human Peripheral blood mononuclear cells (PMBCs) were isolated from whole blood using Ficoll® gradient and human T cells isolated using human CD8+ T Cell Isolation Kit (Miltenyi). Isolated CD8+T were plated at 1×10⁶ cells/mL in X-VIVO 15 medium (Lonza) in a 24 well plate and activated using Dynabeads™ Human T-Activator CD3/CD28 (Thermofisher) for 24 h prior to transduction. Transduction of the cells was performed by adding 20 or 40 μL of lentiviral supernatant (for the CAR and/or Hook) at 10⁷-10⁸ TU/mL with 8 μg/mL of protamine (Sigma) to 1×10⁶ cells, followed by spinoculation at 900 g for 1 h at Room Temperature. After 48 h, the medium of the cells was exchanged and 200 U of IL-2 was added prior to a second round of infection performed with 20-40 μL of lentiviral supernatant (for the CAR and Hook respectively) as described above. The transduced cells were expanded for around 6 days in X-VIVO containing 200 U of IL-2 and the expression of CAR evaluated by FACS and the cytotoxicity activity by xCELLigence (www.aceabio.com/xcelligence-real-time-cell-analysis-rtca-assay-principle/). Note that when the cells were transduced with Hook and CAR, around 5 μg/mL of avidin was added to prevent that the biotin present in the medium induce the detachment of the CAR from the hook in steady state.

xCELLigence to Evaluation CAR-T Cells Cytotoxicity

xCelligence E-Plate® were coated with 10 μg/mL of anti-Human CD40/TNFRSF5 (R&D system) for at least 2 h at 37° C. with 5% CO 2 to capture Raji Target cells in PBS. After one wash with X-VIVO medium, Raji cells in X-VIVO medium were added and incubated overnight at 37° C. with 5% CO 2 to allow their adherence. The following day, CD8+ T cells were added at different ratios effector to target (E:T). Cell index, which is the relative cell impedance, was monitored for at least 60 hours at 37° C. with 5% CO₂.

Flow Cytometry Analysis

Surface expression of scFvCD19 of CAR in T-cells was evaluated using Recombinant Human CD19 fused to human Fc Chimera Protein (R&D system) followed by anti-human PE (BD bioscience). The viability of the cells was assessed using live/dead fixable staining (Invitrogen). The flow cytometry measurements were performed in BD FACSVerse—BD Biosciences and/or MACS Quant Analyzer 10 (Miltenyi).

Results

The CAR in T cells were adapted to the RUSH technology. CARTune technology was developed based on the previous published RUSH system by Boncompain et al, Nature Methods, 2012. This system allows the synchronization of CAR traffic to cell surface, and thus specific CAR induced cell activation. In this system CAR is fused to streptavidin binding Peptide (SBP) that specifically interacts with streptavidin (Str) coupled to an endoplasmic reticulum-resident protein (named the Hook), imposing its ER retention (resting state). This interaction can be reversed by the addition of biotin which will interact with SBP, leading to CAR release and thus traffic to cell surface (activation state). This shall allow CAR-T cell recognition of tumor antigen and subsequent activation and killing.

Preliminary experiments were carried out in primary CD8+T cells co-transduced with lentiviral particles containing the Hook (10⁸ TU/mL) or the scFv-CD19 CAR-2A-YFastPuro (appr. 10⁵ TU/mL) at 1.35:1 (V/V) proportions leading to an excess of Hook due its higher lentiviral titter. In these conditions, it was observed that the cytotoxicity of CAR (anti-CD19-CAR JUNE) was minimal in normal conditions and strongly increased upon biotin addition in a similar manner than the positive control anti-CD19-CAR JUNE (FIG. 4).

It was further evaluated the cytotoxic capacity of other CAR designs, including two first generations CARs based on the protein DNAX-activation protein (DAP) 10 and 12, and the 2^{nd} generation CAR DAP10-CD3 with an additional CD3ζ domain fused to DAP10 (FIG. 5A). SBP was fused to all CARs in their C-terminus to allow their retention in the endoplasmic reticulum (ER) by the streptavidin fused to the hook (FIG. 5A). DAP10 with YINM activation motif or DAP12 with an Immunoreceptor tyrosine-based activation motif (ITAM) were considered as good candidates for CAR based therapies. The rationale behind was that DAP12 coupled with its activating co-receptors (NKG2D, MDL-1, TREM, amongst others) elicits a signal pathway that promotes the activation of immune cells, including natural killer (NK) and CD8⁺T cells, while DAP10 was previously described to elicit NK effector functions against malignant cells (Billadeau, Upshaw et al. 2003, Quatrini, Molfetta et al. 2015). To burst the cytotoxicity of DAP10, CD3ζ with three ITAM domains was used, as it is well known to provide an effective T cell activation signal. In the CD8 T cells, the surface expression of scFv against CD19 tumor antigen was evaluated for all CARs been the CAR JUNE, CAR DAP10 and CAR DAP10-CD3 with highest expression (60-80%) (FIG. 5B). It could be verified that all CARs containing ITAM domains were effective against cancer cells at different ratio effector: target, contrarily to DAP10 (FIG. 6). Interestingly, although the expression of CAR DAP12 was lower, its ability to kill cancer cells was similar to the other CARs, suggesting that a first generation CAR can be as effective as a second generation to target tumor cells. We consider that first generation CARs can be good CAR candidates to tumor targeting with a presumably lower associated toxicity.

1. A chimeric antigen receptor comprising:
a binding domain,
the DAP 10 protein, the DAP 12 protein, or a functional variant thereof, and a hook binding domain.

2. The chimeric antigen receptor according to claim 1, wherein the hook-binding domain is streptavidin-binding peptide, preferably the hook-binding domain is selected from SEQ ID NO: 1, SEQ ID NO: 3, and SEQ ID NO: 9.

3. The chimeric antigen receptor according to claim 1, wherein the binding domain comprises a single-chain Fv antibody or a single-domain antibody.

4. The chimeric antigen receptor according to claim 1, which further comprises at least one further activation domain selected from the CD3-ζ chain, the CD28 cytoplasmic domain, the 4-1BB cytoplasmic domain, the OX40 cytoplasmic domain and the ICOS cytoplasmic domain.

5. A nucleic acid comprising a nucleic acid sequence encoding a chimeric antigen receptor according to claim 1.

6. A nucleic acid according to claim 5 further comprising a nucleic acid sequence encoding a hook protein; optionally wherein the hook protein comprises a streptavidin domain and an endoplasmic reticulum retention signal.

7. A vector system comprising one or more vector comprising:
(a) a nucleic acid comprising a nucleic acid sequence encoding a chimeric antigen receptor according to claim 5, and optionally
(b) a nucleic acid encoding a hook fusion protein, preferably comprising a streptavidin core;
wherein the nucleic acids (a) and (b) are located on the same or on different vectors; optionally
wherein the hook-binding domain of chimeric antigen receptor is a streptavidin-binding domain and the hook fusion protein comprises a streptavidin domain; optionally wherein the hook fusion protein comprises an amino acid sequence corresponding to SEQ ID NO: 6.

8. The vector system of claim 7 wherein the nucleic acids (a) and (b) are located on the same vector and wherein the nucleic acid sequence encoding the hook fusion protein is inserted upstream an IRES sequence or a 2A peptide and the nucleic acid sequence encoding the chimeric antigen receptor is located downstream said IRES sequence or 2A peptide, and
wherein the nucleic acid sequence encoding the hook fusion protein is operably linked to a promoter.

9. The vector system of claim 7, wherein the vector comprises a β2-microglobulin, ubiquitin, MHCI, or MHCII promoter.

10. A viral vector particle system comprising one or more viral vector particle wherein said viral vector particle system comprises a vector system according to claim 7.

11. An isolated host cell comprising the vector system of claim 7.

12. A kit comprising the chimeric antigen receptor of claim 1.

13. The kit according to claim 12, wherein the hook-binding domain of chimeric antigen receptor is a streptavidin-binding domain and the hook protein comprises a streptavidin domain, and which further comprises a streptavidin ligand, preferably the streptavidin ligand is biotin.


SEQUENCE LISTING (16 sequences)

SEQ ID NO: 1 - protein, 38 aa, synthetic construct
  REGION 1..38: note = SBP sequence
MDEKTTGWRGGHVVEGLAGELEQLRARLEHHPQGQREP

SEQ ID NO: 2 - DNA, 114 bp, synthetic construct
  misc_feature 1..114: note = SBP sequence
  source 1..114: mol_type = other DNA
atggacgagaaaaccaccggctggcggggaggccacgtggtggaaggactggccggcgagctggaacagctgcgggccagactggaacaccacccccagggccagagagagccc

SEQ ID NO: 3 - protein, 28 aa, synthetic construct
  REGION 1..28: note = short SBP
GHVVEGLAGELEQLRARLEHHPQGQREP

SEQ ID NO: 4 - protein, 160 aa, Streptomyces avidinii
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 5 - protein, 160 aa, synthetic construct
  REGION 1..160: note = monomeric streptavidin
MDPSKDSKAQVSAAEAGITGTWYNQLGSTFIVTAGADGALTGTYESAVGNAESRYTLTGRYDSAPATDGSGTALGWRVAWKNNYRNAHSATTWSGQYVGGAEARINTQWTLTSGTTEANAWKSTLRGHDTFTKVKPSAASIDAAKKAGVNNGNPLDAVQQ

SEQ ID NO: 6 - protein, 407 aa, synthetic construct
  REGION 1..407: note = Hook sequence
MHRRRSRSCREDQKPVTGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQVDYPYDVPDYAVGPMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 7 - protein, 398 aa, synthetic construct
  REGION 1..398: note = CAR sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPAGEQKLISEEDLGRPLRPVQAQAQSDCSCSTVSPGVLAGIVMGDLVLTVLIALAVYFLGRLVPRGRGAAEAATRKQRITETESPYQELQGQRSDVYSDLNTQRPYYKHVVEGLAGELEQLRARLEHHPQGQREP

SEQ ID NO: 8 - protein, 381 aa, synthetic construct
  REGION 1..381: note = CAR sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPAGEQKLISEEDLGRPQTTPGERSSLPAFYPGTSGSCSGCGSLSLPLLAGLVAADAVASLLIVGAVFLCARPRRSPAQEDGKVYINMPGRGHVVEGLAGELEQLRARLEHHPQGQREP

SEQ ID NO: 9 - protein, 29 aa, synthetic construct
  REGION 1..29: note = short SBP
GGHVVEGLAGELEQLRARLEHHPQGQREP

SEQ ID NO: 10 - DNA, 1143 bp, synthetic construct
  misc_feature 1..1143: note = CAR sequence
  source 1..1143: mol_type = other DNA
atggccttaccagtgaccgccttgctcctgccgctggccttgctgctccacgccgccaggccggatatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcacctgcaggagagcagaagctgatctcagaggaggacctgggccggccacagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggccacgttgttgaaggactggctggggaacttgaacaacttcgtgcacgactggagcatcacccacaaggtcaacgtgaacca

SEQ ID NO: 11 - DNA, 1197 bp, synthetic construct
  misc_feature 1..1197: note = CAR sequence
  source 1..1197: mol_type = other DNA
atggccttaccagtgaccgccttgctcctgccgctggccttgctgctccacgccgccaggccggatatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcacctgcaggagagcagaagctgatctcagaggaggacctgggccggccactccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaacacgttgttgaaggactggctggggaacttgaacaacttcgtgcacgactggagcatcacccacaaggtcaacgtgaaccatga

SEQ ID NO: 12 - DNA, 1500 bp, synthetic construct
  misc_feature 1..1500: note = CAR sequence
  source 1..1500: mol_type = other DNA
atggccttaccagtgaccgccttgctcctgccgctggccttgctgctccacgccgccaggccggatatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcacctgcaggagagcagaagctgatctcagaggaggacctgggccggccacagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggccttaagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaccggtggccacgttgttgaaggactggctggggaacttgaacaacttcgtgcacgactggagcatcacccacaaggtcaacgtgaaccatga

SEQ ID NO: 13 - protein, 499 aa, synthetic construct
  REGION 1..499: note = CAR sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPAGEQKLISEEDLGRPQTTPGERSSLPAFYPGTSGSCSGCGSLSLPLLAGLVAADAVASLLIVGAVFLCARPRRSPAQEDGKVYINMPGRGLKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTGGHVVEGLAGELEQLRARLEHHPQGQREP

SEQ ID NO: 14 - protein, 75 aa, Homo sapiens
QTTPGERSSLPAFYPGTSGSCSGCGSLSLPLLAGLVAADAVASLLIVGAVFLCARPRRSPAQEDGKVYINMPGRG

SEQ ID NO: 15 - protein, 92 aa, Homo sapiens
LRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 16 - protein, 190 aa, synthetic construct
  REGION 1..190: note = DAP10CD3
QTTPGERSSLPAFYPGTSGSCSGCGSLSLPLLAGLVAADAVASLLIVGAVFLCARPRRSPAQEDGKVYINMPGRGLKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR